(19) United States
(12) Patent Application Publication
(10) Pub. No.: US 2025/0362307 A1
(43) Pub. Date: Nov. 27, 2025

(54) PHOTOACTIVE ANTIBODY CONJUGATE

(71) Applicants: Jung-Chi LIAO, Taipei City (TW); SYNCELL (TAIWAN) INC., Taipei City (TW)

(72) Inventors: Jung-Chi LIAO, Taipei City (TW); Hoi Yin CHEUNG, Taipei (TW); Yi-De CHEN, Taipei (TW); Chia-Wen CHUNG, Taipei (TW); Chih-Wei CHANG, Taipei (TW); Hsiao-Jen CHANG, New Taipei City (TW)

(21) Appl. No.: 18/848,699

(22) PCT Filed: Apr. 7, 2023
(86) PCT No.: PCT/US2023/065541

§ 371 Date: Sep. 19, 2024

Related U.S. Application Data
(60) Provisional application No. 63/329,219, filed on Apr. 8, 2022.

(51) Int. Cl. G01N 33/68

(52) U.S. Cl. CPC G01N 33/6875

(57) ABSTRACT

Kits including photoreactive probes and primary subject probes and methods of using the kits. The photoreactive probe can be activated in a selected region of interest by optical radiation, and the activated photoreactive probe allows the primary subject probe to form a covalent bond with molecules in a sample in the selected region of interest. The kits and methods may be useful for analyzing biomolecules in a biological sample.

### CLAIM OF PRIORITY

This application is a 371 national phase application International Application No. PCT/US2023/065541, filed Apr. 7, 2023, which claims priority to U.S. Provisional Patent Application No. 63/329,219 filed on Apr. 8, 2022, titled “PHOTOACTIVE ANTIBODY CONJUGATE,” which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are methods and kits for identifying, tagging, and analyzing biomolecules. Specifically described are photoreactive kits useful for photoactivated and tagging of subsets of biomolecules. The methods and kits may be particularly useful for analyzing biological samples, such as identifying proximal biomolecules in cell or tissue samples.

### BACKGROUND

Cells are composed of different types of biological molecules (biomolecules). The biomolecules in the cells interact with neighbor biomolecules in the subcellular environment to form complexes, organelles, or other assemblies and to carry out various essential cell functions. Characterizing the subcellular environment, within which biomolecules interact with one another, and how the biomolecules function together is very challenging. Biomolecules are small and they exist in a cell environment with tens of millions of other molecules. The interactions between neighboring biomolecules are frequently weak, and many techniques used to study biomolecules disrupt their interactions. While techniques such as yeast two-hybridization assays and more recently proximity labeling have helped advance our understanding of the cell environment, these techniques suffer from various limitations, such as nonspecific binding, slow reaction times, and disruption of the natural cell environment, resulting in false positives and missed interactions. What is needed are better tools for determining naturally occurring biomolecule interactions to address these or other problems.

### SUMMARY OF THE DISCLOSURE

Described herein are systems, kits, and methods for identifying, tagging, and analyzing biomolecules. Specifically described are photoreactive kits useful for photoactivated and tagging of subsets of biomolecules. The methods and kits may be particularly useful for analyzing biological samples, such as identifying proximal biomolecules in cell or tissue samples. These kits may be especially useful for selectively tagging and proximity labeling of biomolecules via selective light illumination through a microscope system.

Described herein are photoreactive kits including a photoreactive probe represented by formula (I): G-C-B (I) wherein the C portion is a single chemical bond or a linker; the B portion includes one to fifty photoreactive moieties and is bound to the C portion, wherein each of the one to fifty photoreactive moieties is derived from a ruthenium-based compound represented by formula (II):

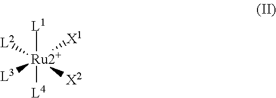

wherein in formula (II): L¹, L², L³, and L⁴ are each independently a ligand; and X¹ and X² are each independently a ligand having a reactive moiety, wherein the reactive moiety is configured for bonding to the C portion; the G portion includes a bait molecule bound to the C portion, wherein the bait molecule is an antibody and configured to conjugate with a first molecule in a sample. These and other embodiments can include a primary subject probe including a detectable tag portion bound to a photoexcitable subject moiety, wherein, when the photoreactive probe is photoactivated at either a wavelength ranging from 700 nm to 1100 nm with a two-photon light source or a wavelength ranging from 200 nm to 1100 nm with a single light source, and the primary subject probe is acted upon by the photoactivated probe to form a photoexcited primary subject probe, the photoexcited primary subject probe is configured to form a covalent bond with a target molecule in the sample.

In these and other embodiments, the photoreactive kit may include wherein X¹ and X² are each independently selected from the group consisting of 3-ethynylpyridine, 3-(bromomethyl)pyridine, maleimide, 4′-methyl-4-carboxybipyridine-N-succinimidyl ester, nicotinaldehyde, 1-(4-(pyridin-3-yl)-1H-1,2,3-triazol-1-yl) ethanone, 4-pentynenitrile, and 4-aminobutyne.

In these and other embodiments, the photoreactive kit may include wherein L¹ and L² are joined to form a first bidentate ligand and L³ and L⁴ are joined to form a second bidentate ligand, wherein the first bidentate ligand and the second bidentate ligand are independently selected from the group consisting of 2,2′-bipyridyl (bpy), 4,4′-dicyano-5,5′-dimethyl-2,2′-bipyridine (CN-Me-bpy), 4,4′-dimethyl-2,2′-bipyridine (dmb), 4,4′-di-tert-butyl-2,2′-bipyridine (dbpy), 4,4′,5,5′-tetramethyl-2,2′-bipyridine (tmb), 2-phenylpyridine (ppy), 6-bromo-2,2′-bipyridine, 6,6′-dibromo-2,2′-bipyridine, 5-bromo-2,2′-bipyridine, 6-amino-2,2′-bipyridine, 6,6′-diamino-2,2′-bipyridine, 2,2′-bipyridine-6-carbonitrile, 2,2′-bipyridine-6,6′-bis(carbonitrile), 2,2′-bipyridine-6-carboxylic acid, 2,2′-bipyridine-6,6′-dicarboxylic acid, and biquinoline.

In these and other embodiments, the photoreactive kit may include wherein the ruthenium-based compound of formula (II) is one of:

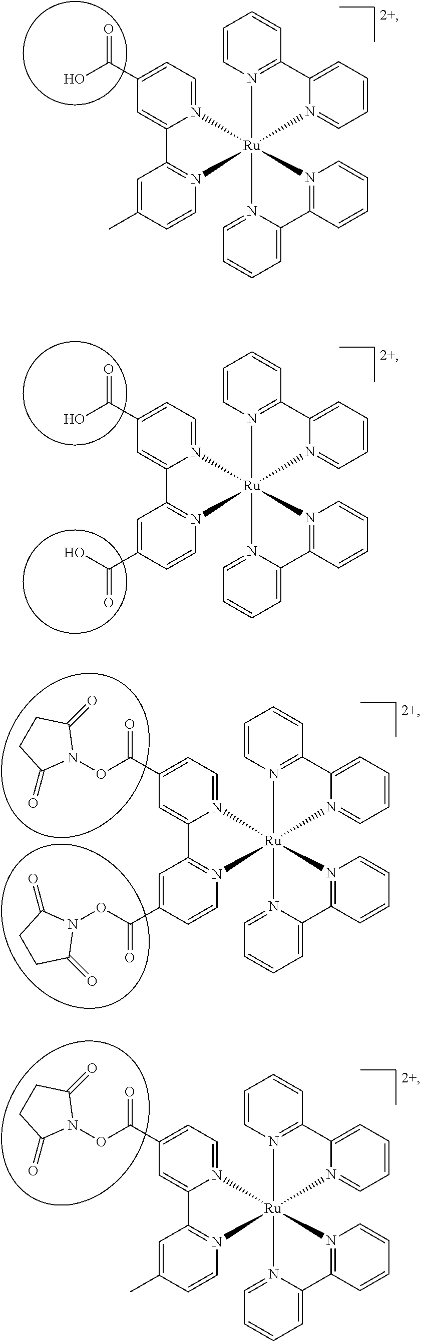

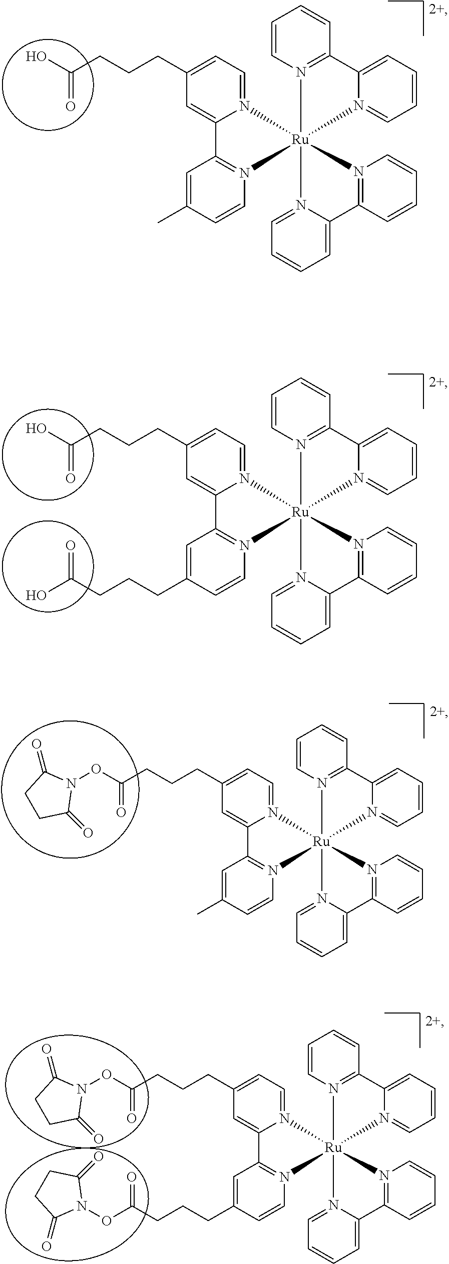

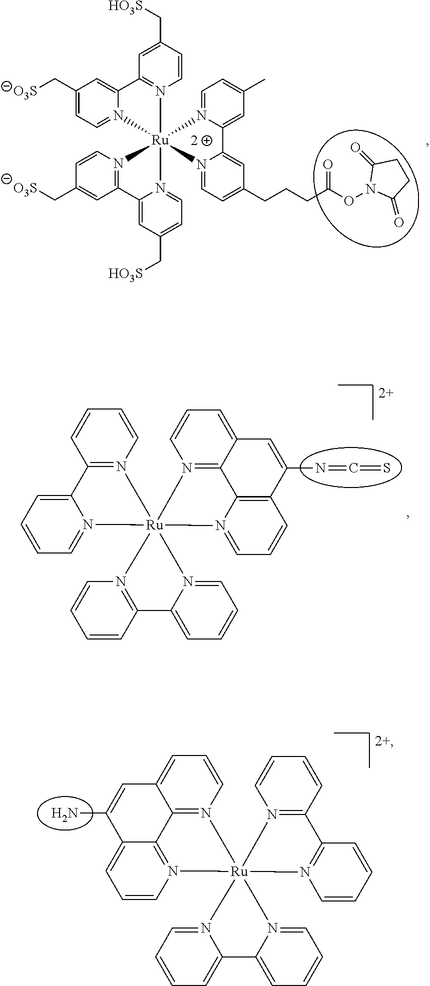

of a derivative thereof.

In these and other embodiments, the photoreactive kit may include wherein the photoreactive moiety includes the moiety of

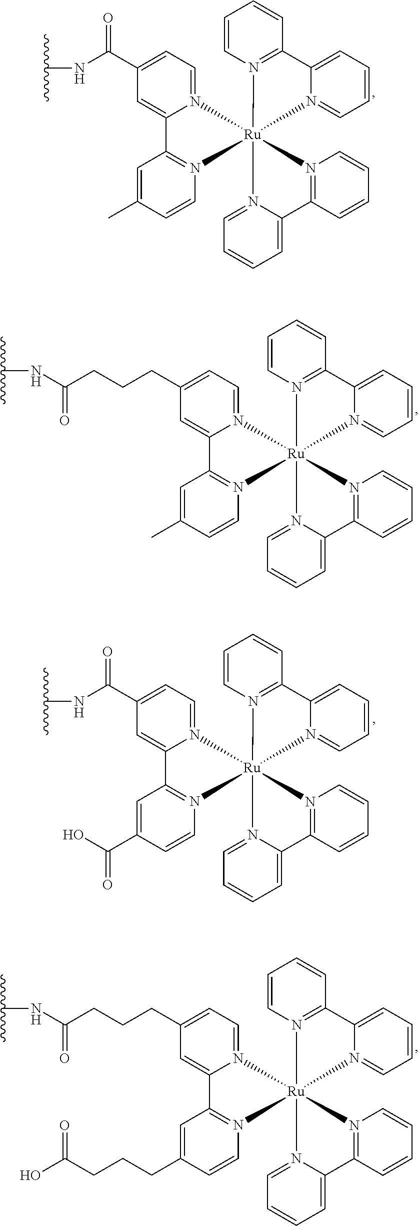

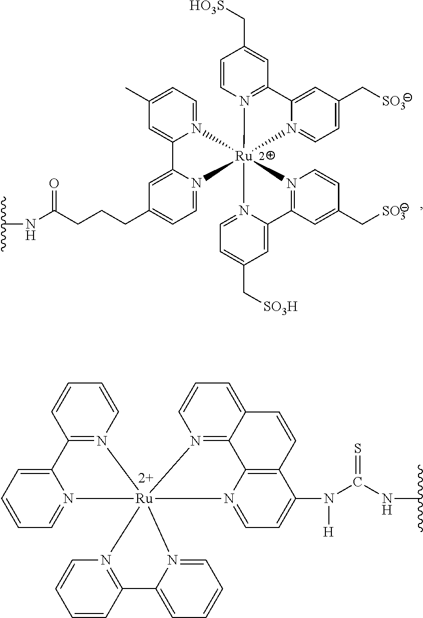

or a derivative thereof.

In these and other embodiments, the linker can include the moiety of

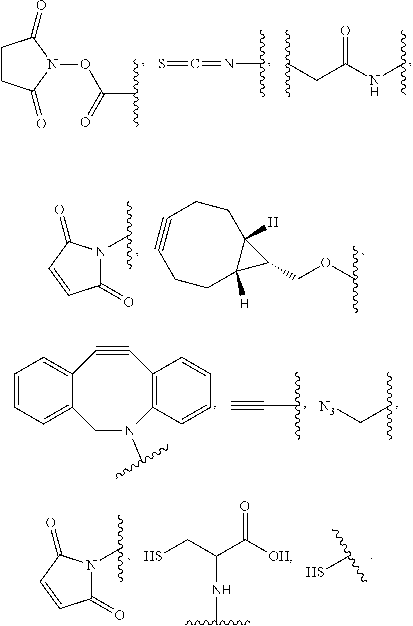

In these and other embodiments, the photoreactive kit may include wherein the linker includes at least one of an amino acid, (PEG) n, an oligonucleotide, or a peptide, wherein n is an integer from 1 to 20.

In these and other embodiments, the photoreactive kit may include wherein when the photoreactive probe is photoactivated at a wavelength ranging from 700 nm to 1100 nm with a two-photon light source, and the primary subject probe is acted upon by the photoactivated probe to form a photoexcited primary subject probe, the photoexcited primary subject probe is configured to form a covalent bond with a target molecule in the sample. In these and other embodiments, the photoreactive kit may include further wherein when the photoreactive probe is photoactivated at a wavelength ranging from 300 nm to 800 nm with a single light source, and the primary subject probe is acted upon by the photoactivated probe to form a photoexcited primary subject probe, the photoexcited primary subject probe is configured to form a covalent bond with a target molecule in the sample.

In these and other embodiments, the photoreactive kit may include wherein the detectable tag portion is at least one of a biotin derivative, a digoxigenin tag, a CLIP-tag, a HaloTag, a SNAP-tag, an oligonucleotide, a peptide tag, and a click chemistry tag, and the click chemistry tag includes an alkyne-based or azide-based moiety.

In these and other embodiments, the photoreactive kit may include wherein the photoexcitable subject moiety is at least one of

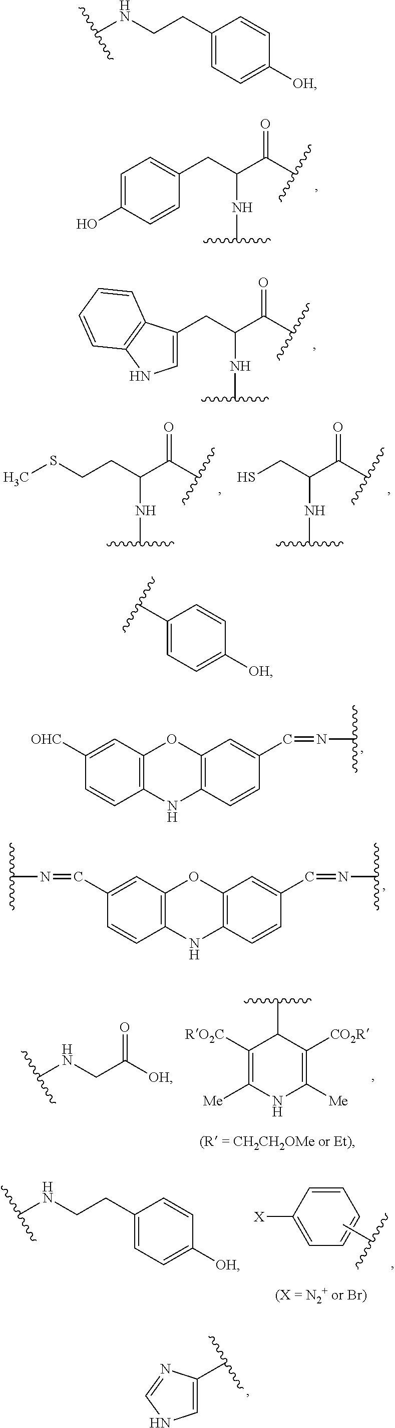

and a derivative thereof.

In these and other embodiments, the photoreactive kit may include wherein the primary subject probe is desthiobiotin-phenol or biotin-phenol.

In these and other embodiments, the photoreactive kit may include wherein the photoexcitable subject moiety is

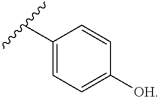 [SMILES: *c1ccc(O)cc1]

In these and other embodiments, the photoreactive kit may include wherein the detectable tag portion is at least one of a biotin derivative, a click chemistry tag, a CLIP-tag, a digoxigenin tag, a HaloTag, an oligonucleotide, a peptide tag, a SNAP-tag and the photoreactive moiety includes the moiety of

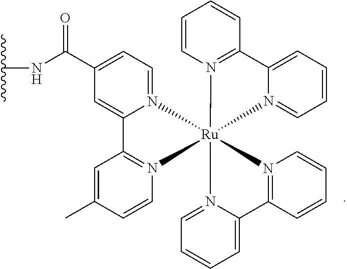

In these and other embodiments, the photoreactive kit may include further including a connector, wherein the connector is conjugatable with the detectable tag portion of the primary subject probe. In these and other embodiments, the photoreactive kit may include wherein the connector is a fluorescent connector. In these and other embodiments, the photoreactive kit may include further including a tag-enzyme complex, wherein the tag-enzyme complex is conjugatable with the connector, and further wherein the enzyme of the tag-enzyme complex includes peroxidase. In these and other embodiments, the photoreactive kit may include a connector, wherein the connector is conjugatable with the detectable tag portion of the primary subject probe; a tag-enzyme complex, wherein the tag of the tag-enzyme complex is conjugatable with the connector, and further wherein the enzyme of the tag-enzyme complex includes peroxidase; and an additional subject probe configured to form an additional subject probe covalent bond with the target molecule by catalytic activity of the peroxidase of the tag-enzyme complex. In these and other embodiments, the photoreactive kit may include wherein the additional subject probe is the same as or different from the primary subject probe and includes an additional subject probe tag portion and an additional subject probe subject moiety. In these and other embodiments, the photoreactive kit may include wherein the connector is a fluorescent connector.

In these and other embodiments, the photoreactive kit may include wherein a concentration of the photoreactive probe ranges from 0.1 ug/mL to 100 ug/mL and a concentration of the primary subject probe ranges from 1 uM to 20 mM.

Also described herein are photoreactive kits including a photoreactive probe represented by formula (I): G-C-B (I) wherein the C portion is a single chemical bond or a linker; the B portion includes at least one photoreactive moiety bound to the C portion; and the G portion includes a bait molecule bound to the C portion. In these and other embodiments, the photoreactive kit may include a primary subject probe including a detectable tag portion bound to a photoexcitable subject moiety, wherein the bait molecule of the photoreactive probe is configured to conjugate with a first molecule in a sample, and wherein, when the photoreactive probe is photoactivated and the primary subject probe is acted upon by the photoactivated probe to form a photoexcited primary subject probe, the photoexcited primary subject probe is configured to form a covalent bond with a target molecule in the sample.

In these and other embodiments, the photoreactive kit may include wherein the bait molecule includes at least one of an antibody, avidin, neutravidin, streptavidin, another biotin-binding protein, a CLIP-tag, a HaloTag, a SNAP-tag, another self-labeling protein tag, a DNA or RNA fluorescent in situ hybridization (FISH) probe, another RNA molecule, another nucleic acid molecule, protein A, protein G, protein L, protein A/G, protein A/G/L, another immunoglobulin binding peptide, a drug, or another small molecule.

In these and other embodiments, the photoreactive kit may include wherein the photoreactive moiety is at least one of riboflavin, lumiflavin, another flavin derivatives, fluorescein or a derivative thereof, methylene blue or a derivative thereof, miniSOG photosensitized protein, Killer Red photosensitized protein, another photosensitized protein, pterin or a derivative thereof, a ruthenium-based photocatalyst, and Rose Bengal or a derivative thereof.

In these and other embodiments, the photoreactive kit may include wherein the bait molecule is an antibody and a number of the photoreactive moieties are bound to the antibody through the C portion, wherein the number ranges from 1 to 50.

In these and other embodiments, the photoreactive kit may include wherein the photoreactive moiety is configured to allow the primary subject probe to form the covalent bond with the molecule of the sample.

In these and other embodiments, the photoreactive kit may include wherein the photoreactive moiety is derived from a ruthenium-based compound represented by formula (II):

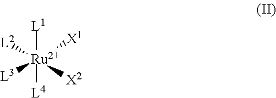

wherein in formula (II): L¹, L², L³, and L⁴ are each independently a ligand; and X¹ and X² are each independently a ligand, wherein at least one of X¹ and X² has linking region, wherein the at least one linking region is bound to the C portion of the photoreactive probe.

In these and other embodiments, the photoreactive kit may include wherein X¹ and X² are each independently selected from the group consisting of 3-ethynylpyridine, 3-(bromomethyl)pyridine, maleimide, 4′-methyl-4-carboxybipyridine-N-succinimidyl ester, nicotinaldehyde, 1-(4-(pyridin-3-yl)-1H-1,2,3-triazol-1-yl) ethanone, 4-pentynenitrile, and 4-aminobutyne.

In these and other embodiments, the photoreactive kit may include wherein L¹ and L² are joined to form a first bidentate ligand and L³ and L⁴ are joined to form a second bidentate ligand, wherein the first bidentate ligand and the second bidentate ligand are independently selected from the group consisting of 2,2′-bipyridyl (bpy), 4,4′-dicyano-5,5′-dimethyl-2,2′-bipyridine (CN-Me-bpy), 4,4′-dimethyl-2,2′-bipyridine (dmb), 4,4′-di-tert-butyl-2,2′-bipyridine (dbpy), 4,4′,5,5′-tetramethyl-2,2′-bipyridine (tmb), 2-phenylpyridine (ppy), 6-bromo-2,2′-bipyridine, 6,6′-dibromo-2,2′-bipyridine, 5-bromo-2,2′-bipyridine, 6-amino-2,2′-bipyridine, 6,6′-diamino-2,2′-bipyridine, 2,2′-bipyridine-6-carbonitrile, 2,2′-bipyridine-6,6′-bis(carbonitrile), 2,2′-bipyridine-6-carboxylic acid, 2,2′-bipyridine-6,6′-dicarboxylic acid, and biquinoline.

In these and other embodiments, the photoreactive kit may include wherein the ruthenium-based compound of formula (II) is any of

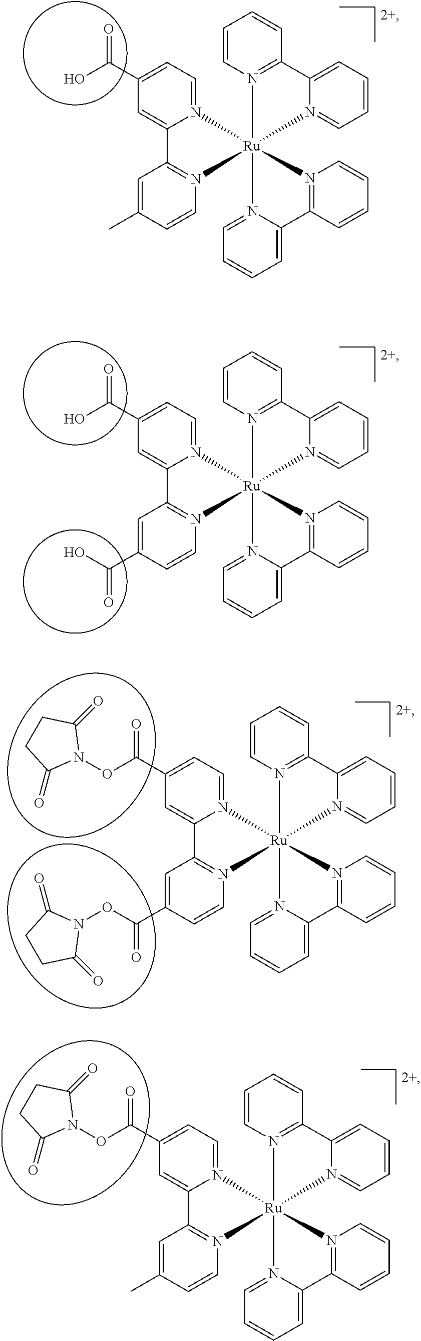

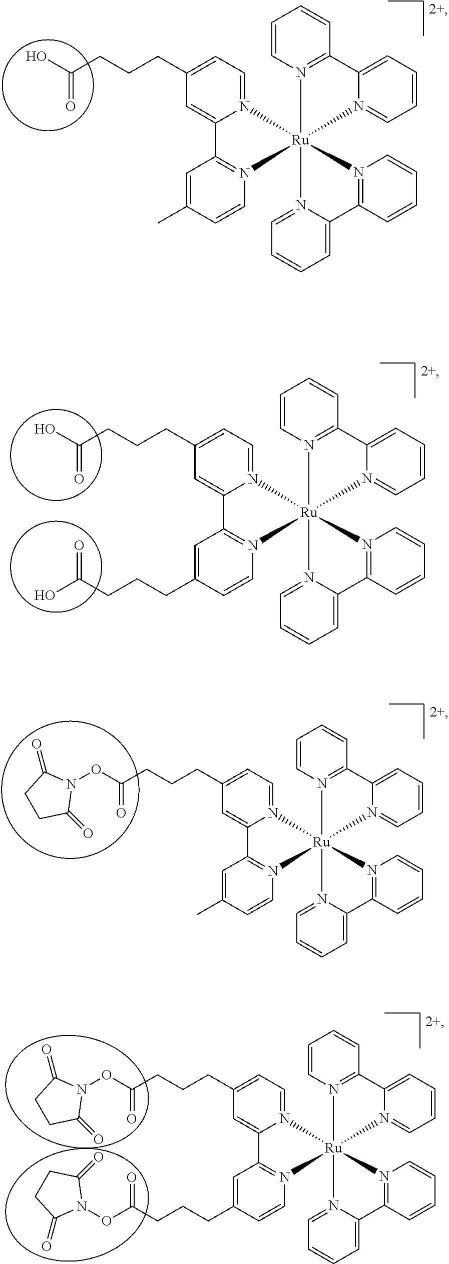

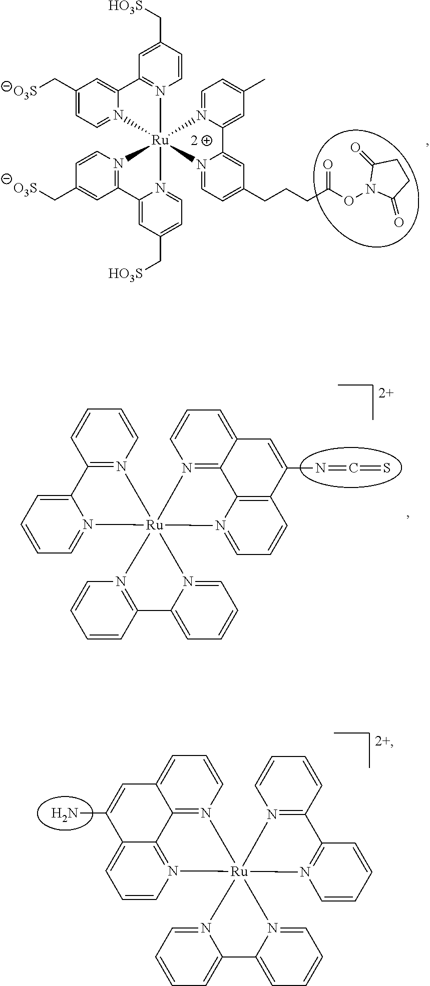

and derivatives thereof.

In these and other embodiments, the photoreactive kit may include wherein the photoreactive moiety includes the moiety of

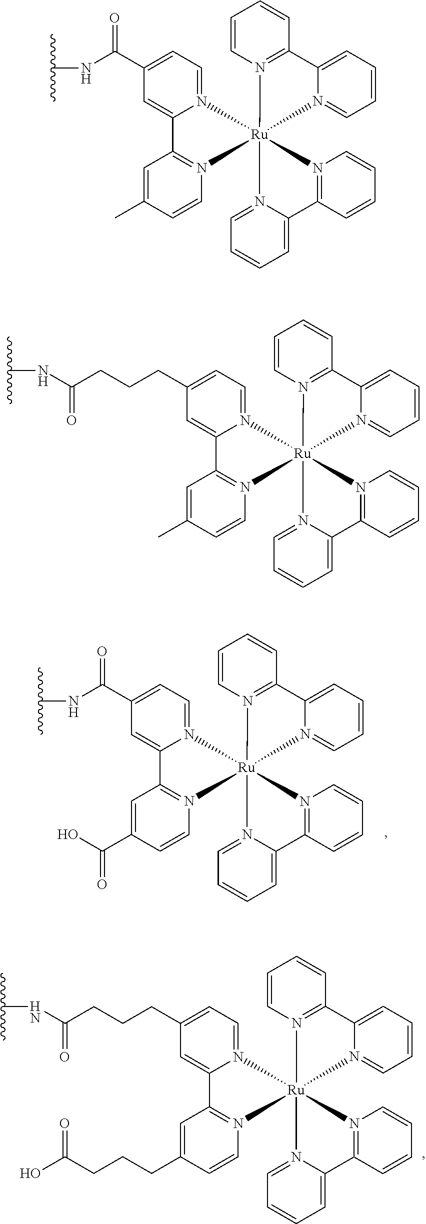

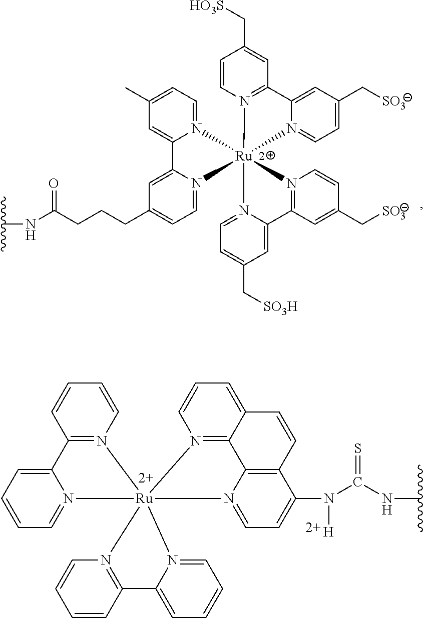

or a derivative thereof.

In these and other embodiments, the photoreactive kit may include wherein the linker includes the moiety of

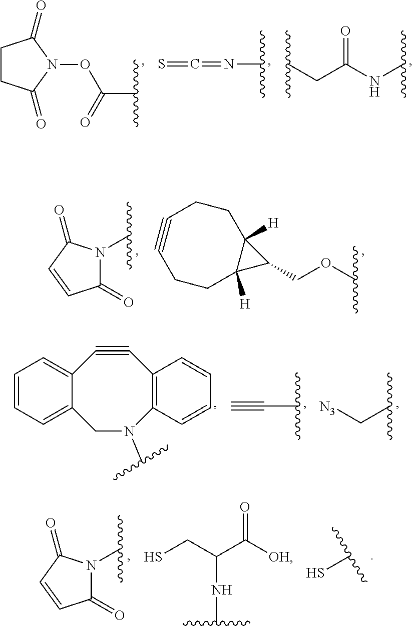

In these and other embodiments, the photoreactive kit may include wherein the linker includes at least one of an amino acid, (PEG) n, an oligonucleotide, or a peptide, wherein n is an integer from 1 to 20.

In these and other embodiments, the photoreactive kit may include wherein the photoreactive moiety is activatable at a wavelength ranging from 200 nm to 1100 nm with a light source so as to allow the primary subject probe to form the covalent bond with the target molecule in the sample.

In these and other embodiments, the photoreactive kit may include wherein the photoreactive moiety is activatable at a wavelength ranging from 700 nm to 1100 nm with a two-photon light source so as to allow the primary subject probe to form the covalent bond with the target molecule in the sample. In these and other embodiments, the photoreactive kit may include wherein the photoreactive moiety is activatable at a wavelength ranging from 300 nm to 800 nm with a light source so as to allow the primary subject probe to form the covalent bond with the target molecule in the sample.

In these and other embodiments, the photoreactive kit may include wherein the photoreactive moiety is activatable at a wavelength ranging from 700 m to 1100 nm with a two-photon light source so as to allow the primary subject probe to form the covalent bond with the target molecule in the sample.

In these and other embodiments, the photoreactive kit may include wherein the detectable tag portion is at least one of a biotin derivative, a CLIP-tag, a digoxigenin tag, a HaloTag, an oligonucleotide, a peptide tag, a SNAP-tag, and a click chemistry tag, and the click chemistry tag includes an alkyne-based or azide-based moiety.

In these and other embodiments, the photoreactive kit may include wherein the subject moiety is one or more of

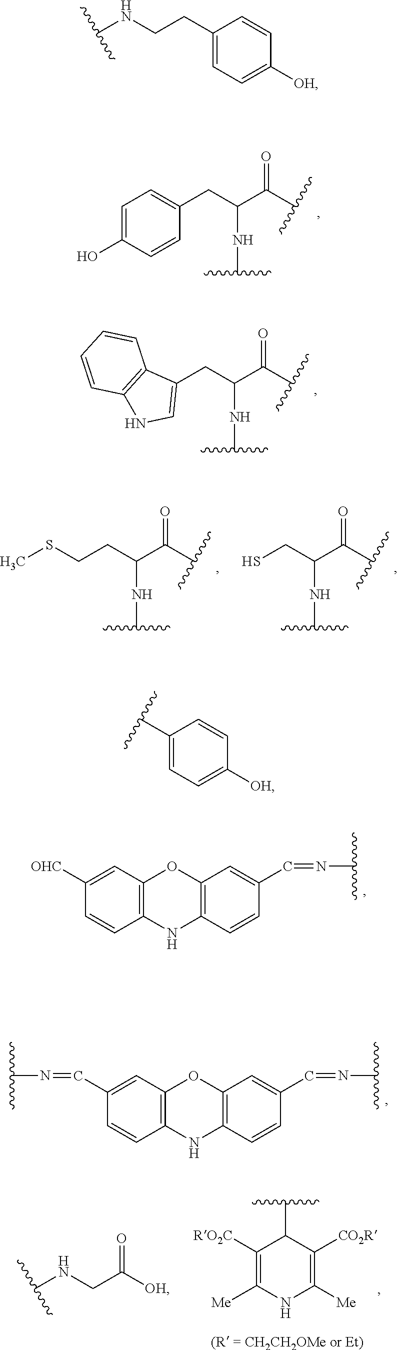

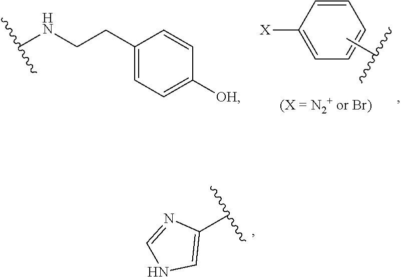

and a derivative thereof.

In these and other embodiments, the photoreactive kit may include wherein the primary subject probe is desthiobiotin-phenol or biotin-phenol.

In these and other embodiments, the photoreactive kit may include wherein the photoexcitable subject moiety is

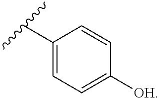 [SMILES: *c1ccc(O)cc1]

In these and other embodiments, the photoreactive kit may include wherein the detectable tag portion is at least one of a biotin derivative, a click chemistry tag, a CLIP-tag, a digoxigenin tag, a HaloTag, an oligonucleotide, a peptide tag, a SNAP-tag and the photoreactive moiety includes the moiety of

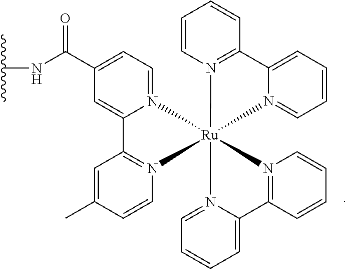

In these and other embodiments, the photoreactive kit may include further including a connector, wherein the connector is conjugatable with the detectable tag portion of the primary subject probe. In these and other embodiments, the photoreactive kit may include wherein the connector is a fluorescent connector. In these and other embodiments, the photoreactive kit may include further including a tag-enzyme complex, wherein the tag-enzyme complex is conjugatable with the connector, and further wherein the enzyme of the tag-enzyme complex includes peroxidase.

In these and other embodiments, the photoreactive kit may include further including a connector, wherein the connector is conjugatable with the detectable tag portion of the primary subject probe; a tag-enzyme complex, wherein the tag of the tag-enzyme complex is conjugatable with the connector, and further wherein the enzyme of the tag-enzyme complex includes peroxidase; and an additional subject probe configured to form an additional subject probe covalent bond with the target molecule by catalytic activity of the peroxidase of the tag-enzyme complex.

In these and other embodiments, the photoreactive kit may include wherein the additional subject probe is different from the primary subject probe and includes an additional subject probe tag portion and an additional subject probe subject moiety. In these and other embodiments, the photoreactive kit may include wherein the connector is a fluorescent connector.

In these and other embodiments, the photoreactive kit may include wherein a concentration of the photoreactive probe ranges from 0.1 ug/mL to 100 ug/mL and a concentration of the primary subject probe ranges from 1 uM to 20 mM.

Also described herein are methods for photoreactive labeling. These and other methods can include one or more of the steps of delivering a plurality of photoreactive probes of a photoreactive kit to a sample; non-covalently conjugating the bait molecules of a first portion of the photoreactive probe to a plurality of first molecules in the sample; delivering a plurality of primary subject probes of the photoreactive kit as described herein to the sample; selectively illuminating a selected region of interest of the sample with optical radiation to thereby activate the photoreactive moieties of a plurality of photoreactive probes to form a plurality of photoactivated photoreactive probes in the selected region; photoexciting, with the plurality of photoactivated photoreactive probes, the photoexcitable subject moieties of the primary subject probes to form a plurality of photoexcited primary subject probes with photoexcited subject moieties; and forming covalent bonds between the plurality of photoexcited primary subject probes and a plurality of target molecules in the selected region of interest in the sample to thereby bind the plurality of primary subject probes to the plurality of target molecules.

In these and other methods wherein the step of non-covalently conjugating the bait molecules of a first portion of the photoreactive probe to a plurality of first molecules in the sample leaves a second portion of the photoreactive probe unconjugated, the method may further includes a step of: removing the unconjugated photoreactive probe from the sample.

These and other methods can further include one or more of the steps of: delivering a plurality of connectors to the sample, and conjugating the plurality of connectors to the detectable tag portions of the plurality of primary subject probes.

In these and other methods wherein the plurality of connectors comprise fluorescent connectors, the method can further include one or more of the steps of: detecting, in the sample, a location of the plurality of fluorescent connectors, and thereby identifying a location of the plurality of primary subject probes and the plurality of target molecules covalently bound thereto.

In these and other methods the step of photoexciting can further include the step of photoexciting the primary subject probes to form a plurality of photoexcited primary subject probes each having a free radical, and wherein the step of forming covalent bonds includes forming a covalent bond between each of the plurality of photoexcited primary subject probes and an amino acid in each of the plurality of target molecule in the selected region of interest in the sample.

In these and other methods the step of forming covalent bonds can include forming a covalent bond between each of the plurality of photoexcited primary subject probes and an amino acid in each of the plurality of target molecule in the selected region of interest in the sample.

In these and other methods the amino acid can be selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

In these and other methods can further include one or more of the steps of: delivering the connector of the photoreactive kit as described herein to the sample, wherein the connector is conjugatable with the detectable tag portion of the primary subject probe; delivering the tag-peroxidase complex of the photoreactive kit as described herein to the sample, wherein the tag of the tag-enzyme complex is conjugatable with the connector, and further wherein the enzyme of the tag-enzyme complex includes peroxidase; delivering an additional subject probe of the photoreactive kit as described herein to the sample, wherein the additional subject probe is configured to form an additional subject probe covalent bond with the target molecule by catalytic activity of the peroxidase of the tag-enzyme complex; and conjugating the tag-peroxidase complex to the connector wherein the tag-peroxidase to catalyze the additional subject probe to form a covalent bond between the additional subject probe and the sample.

In these and other methods the tag-peroxidase complex can activate the additional subject probe to have a free radical at the subject moiety of the additional subject probe and form the covalent bond between the subject moiety of the additional subject probe and a tyrosine of the sample.

Also described herein are analytical methods including one or more of the steps of delivering a plurality of photoreactive probes of the photoreactive kit as described herein to the sample; non-covalently conjugating the bait molecules of a first portion of the photoreactive probe to a plurality of first molecules in the sample; delivering a plurality of primary subject probes of the photoreactive kit as described herein to the sample; and illuminating the sample from an imaging lighting source of an image-guided system; imaging the illuminated sample with a camera; acquiring with the camera at least one image of subcellular morphology of the sample in a first field of view; processing the at least one image and determining a region of interest in the sample based on the processed image; obtaining coordinate information of the region of interest; and according to coordinate information, selectively illuminating the region of interest with optical radiation to activate the photoreactive moiety, wherein the activated photoreactive moiety allows the primary subject probe to form a covalent bond with the sample in the region of interest.

These and other methods can include illuminating a region for 10 us/pixel to 200 us/pixel, for 25 us/pixel to 400 us/pixel, for 50 us/pixel to 300 us/pixel, for 75 us/pixel to 200 us/pixel, or for 400 us/pixel to 5000 us/pixel.

In these and other methods the step of selectively illuminating includes illuminating with a power intensity of from 1 μW to 300 mW. In these and other methods the step of selectively illuminating includes illuminating a zone defined by point spread function.

These and other methods can further include a step of conjugating a connector with the primary subject probe and detectably proximity labeling neighbors proximal the target molecule with detectable label activity.

In these and other methods the step of detectably proximity labeling can include proximity labeling a region, less than 5 um, less than 2 um, less than 1 um, less than 500 nm, less than 300 nm, less than 200 nm, less than 100 nm, less than 50 nm, or less than 20 nm in diameter.

In these and other methods, the connector can include a catalytic label.

In these and other methods, a sample can include at least one, at least 100, at least 1000, or at least 10,000 live or fixed cells. In these and other methods, a sample can include fixed cells, fixed tissues, cell extracts, or tissue extracts.

These and other methods can include a step of subjecting the selectively illuminated sample to mass spectrometry analysis or sequencing analysis.

In these and other methods, the activated photoactive moiety can active the primary subject probe to have a free radical and form the covalent bond with an amino acid of the sample in the selected region of interest.

Also described herein are mass spectrometry-implemented methods for processing a sample to predict a biomarker. Described herein are methods that can include one or more of the steps of dividing a sample into a photolabeling sample group and a non-labeling sample group; delivering the photoreactive probe and primary subject probe as described herein to the photolabeling sample group and the non-labeling sample group; selectively illuminating a selected region of interest of the photolabeling sample group and keeping the non-labeling sample group in the dark, wherein the illuminating step allows the primary subject probe to form a covalent bond with the sample; extracting a plurality of the probe-bound proteins from the photolabeling sample group and the non-labeling sample group through an affinity precipitation between the primary subject probe and a plurality of affinity beads; subjecting the extracted proteins to mass spectrometry analysis; calculating a relative quantification value of an individual protein in an identified protein list between the photolabeling sample group and the non-labeling sample group according to an intensity value of a peptide fragment of the individual protein; determining a threshold of the relative quantification value between the photolabeling sample group and the non-labeling sample group; and upon determining the threshold, predicting at least one biomarker corresponding to the relative quantification value of the individual protein over the threshold.

These and other methods can further include a step of non-covalently conjugating the bait molecule to a target molecule in the samples.

In these and other methods the step of selectively illuminating the selected region of interest can further include a step of activating the photoreactive moiety in the selected region so as to allow the activated photoreactive moiety to allow the primary subject probe to form the covalent bond with the sample in the selected region of interest.

These and other methods can further include a step of delivering the bait molecule of the photoreactive probe to the sample.

These and other methods can further include a step of removing unconjugated photoreactive probe from the sample so as to allow the photoreactive probe to guide the selectively illumination on the selected region of interest.

These and other methods can further include a step of delivering the connector of the photoreactive kit as described herein to the sample and conjugating the connector to the primary subject probe through the affinity between the connector and the primary subject probe.

In these and other methods, the connector is a fluorescent connector for identifying the location of the sample covalently bound with the primary subject probe.

In these and other methods, the activated photoreactive moiety can activate the primary subject probe to have a free radical and form the covalent bond with an amino acid of the sample in the selected region of interest.

In these and other methods, the amino acid can be selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

These and other methods can further include a step of delivering the tag-peroxidase of the photoreactive kit as described herein to the sample and conjugating the tag-peroxidase to the connector so as to allow the tag-peroxidase to catalyze the additional subject probe to form a covalent bond between the additional subject probe and the sample.

In these and other methods, the tag-peroxidase can activate the additional subject probe to have a free radical at the subject moiety of the additional subject probe and form the covalent bond between the subject moiety of the additional subject probe and a tyrosine of the sample.

Also described herein are mass spectrometry-implemented methods for processing a photo-labeled sample to identify a biomarker list. Described herein are methods including one or more of the steps of obtaining a sample; delivering the photoreactive kit as described herein to the sample; selectively illuminating a selected region of interest of the biological sample so as to allow the primary subject probe to label a protein of the sample at the selected region of interest; extracting a plurality of the probe-labeled protein from the sample through an affinity precipitation between the primary subject probe and a plurality of affinity beads; subjecting the extracted proteins to mass spectrometry analysis; and identifying the extracted proteins of the sample.

These and other methods can further include a step of calculating an intensity value of a peptide fragment of each protein from an identified protein list of the sample.

These and other methods can further include a step of ranking the identified protein list according to the intensity value of each protein.

These and other methods can further include a step of non-covalently conjugating the bait molecule to a target molecule in the samples.

In these and other methods, the step of selectively illuminating the selected region of interest can further include a step of activating the photoreactive moiety in the selected region so as to allow the activated photoreactive moiety to allow the primary subject probe to form the covalent bond with the sample in the selected region of interest.

These and other methods can further include a step of delivering the bait molecule of the photoreactive probe to the sample.

These and other methods can further include a step of removing unconjugated photoreactive probe from the sample so as to allow the photoreactive probe to guide the selectively illumination on the selected region of interest.

These and other methods can further include a step of delivering the connector of the photoreactive kit as described herein to the sample and conjugating the connector to the primary subject probe through the affinity between the connector and the primary subject probe.

In these and other methods, the connector can be a fluorescent connector configured for identifying the location of the sample covalently bound with the primary subject probe.

In these and other methods, the activated photoreactive moiety can activate the primary subject probe to have a free radical and form the covalent bond with an amino acid of the sample in the selected region of interest. In these and other methods, the amino acid can be selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

These and other methods can further include a step of delivering the tag-peroxidase of the photoreactive kit as described herein to the sample and conjugating the tag-peroxidase to the connector so as to allow the tag-peroxidase to catalyze the additional subject probe to form a covalent bond between the additional subject probe and the sample.

In these and other methods, the tag-peroxidase can activate the additional subject probe to have a free radical at the subject moiety of the additional subject probe and form the covalent bond between the subject moiety of the additional subject probe and a tyrosine of the sample.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 shows a schematic depiction of a system useful for photoselective spatial tagging and proximity labeling of cells on a substrate.

FIG. 2A shows a schematic illustration of a photoreactive probe with a tag portion and a subject moiety.

FIG. 2B shows a schematic illustration of a subject probe with a tag portion and a subject moiety.

FIG. 2C shows an example of a subject probe with a desthiobiotin tag portion and a phenol subject moiety.

FIG. 2D schematically illustrates a subject probe with a tag portion and a reactive phenolic moiety.

FIG. 2E illustrates interactions of a photoreactive probe and subject probe to effect labeling of a neighboring protein using the subject probe shown in FIG. 2D.

FIGS. 2F-2G schematically illustrates steps in biomolecule proximity tagging in a target region using the probes shown in FIGS. 2A and 2B. FIG. 2F schematically illustrates binding a photoreactive probe to a first molecule.

FIG. 2G schematically illustrates light driven photoreactive probe activation and photoreactive probe binding to neighboring biomolecules after binding the photoreactive probe to the first molecule, as shown in FIG. 2F.

FIG. 2H schematically illustrates a proximity labeling system that can be used to label biomolecules using the probes shown in FIGS. 2A and 2B.

FIG. 2I shows a schematic illustration comparing different methods of labelling biomolecules in a small region of interest (ROI). The top right-hand panel illustrates direct photochemical labeling and the bottom panels illustrate photo-assisted enzymatic labeling using a photoreactive probe and a subject probe as described herein to label a protein of interest.

FIGS. 3A-3N show examples of ruthenium-based photoreactive moieties that can be used in the photoreactive probes described herein. FIG. 3A shows an example of a ruthenium-based photoreactive moiety that can be used in the photoreactive probes and methods described herein.

FIG. 3B shows another example of a ruthenium-based photoreactive moiety.

FIG. 3C shows another example of a ruthenium-based photoreactive moiety.

FIG. 3D. shows another example of a ruthenium-based photoreactive moiety.

FIG. 3E shows another example of a ruthenium-based photoreactive moiety.

FIG. 3F shows another example of a ruthenium-based photoreactive moiety.

FIG. 3G shows another example of a ruthenium-based photoreactive moiety.

FIG. 3H shows another example of a ruthenium-based photoreactive moiety.

FIG. 3I shows another example of a ruthenium-based photoreactive moiety.

FIG. 3J shows another example of a ruthenium-based photoreactive moiety.

FIG. 3K shows another example of a ruthenium-based photoreactive moiety.

FIG. 3L shows another example of a ruthenium-based photoreactive moiety.

FIG. 3M shows another example of a ruthenium-based photoreactive moiety.

FIG. 3N shows another example of a ruthenium-based photoreactive moiety.

FIG. 3O shows an example of a Rose Bengal moiety and derivatives thereof that can be used in the photoreactive probes and methods described herein.

FIGS. 3P-3Q show examples of fluorescein derivatives that can be used in the photoreactive probes and methods described herein.

FIG. 3R show an example of a methylene blue derivative that can be used in the photoreactive probes and methods described herein.

FIGS. 3S-3T show examples of lumiflavin derivatives that can be used in the photoreactive probes and methods described herein.

FIGS. 3U-3V show examples of riboflavin and flavin derivatives that can be used in the photoreactive probes and methods described herein.

FIGS. 3W and 3X show examples of pterin derivatives that can be used in the photoreactive probes and methods described herein.

FIG. 3Y shows another example of a ruthenium-based photoreactive moiety.

FIG. 3Z shows another example of a ruthenium-based photoreactive moiety.

FIG. 3AA shows another example of a ruthenium-based photoreactive moiety.

FIG. 3AB shows another example of a ruthenium-based photoreactive moiety.

FIG. 3AC shows another example of a ruthenium-based photoreactive moiety.

FIG. 3AD shows another example of a ruthenium-based photoreactive moiety.

FIG. 3AE shows another example of a ruthenium-based photoreactive moiety.

FIG. 3AF shows another example of a ruthenium-based photoreactive moiety.

FIG. 3AG shows another example of a ruthenium-based photoreactive moiety.

FIG. 3AH shows another example of a ruthenium-based photoreactive moiety.

FIG. 3AI shows another example of a ruthenium-based photoreactive moiety.

FIGS. 4A-4L show examples of linker moieties that can be used in the photoreactive probes described herein.

FIGS. 5A-5E show examples of tags that can be used in the subject probes described herein. FIGS. 5A-5E show click chemistry molecules.

FIGS. 5F-5H show biotin derivatives that can be used in the probes and methods described herein.

FIG. 5I shows a digoxigenin molecule that can be used in the probes and methods described herein.

FIG. 5J shows an example of a peptide tag that can be used in the probes and methods described herein.

FIG. 5K shows an example of a SNAP-tag that can be used in the probes and methods described herein.

FIGS. 6A-6L shows examples of subject moieties that can be used in the subject probes described herein.

FIG. 7 schematically illustrates conjugation of Ru(bpy)₃²⁺ onto an antibody via NHS-ester based amide coupling to form a photoreactive probe.

FIG. 8 shows experimental results of antibody-based ruthenium (Ab-Ru) targeted photo-labeling (confocal imaging of nucleolar markers (magenta; first panel in the XY-axis and Z-axis results), antibody-based ruthenium (red; second panel from the left in the XY-axis and second panel down in the Z axis), photo (2P) labeled signals: desthiobiotin (green; third panel from the left in the XY-axis and third panel down in the Z axis); scale bar: 20 μm).

FIG. 9 shows that small regions of interest (nucleoli) are accurately photo-labeled using an Ab-Ru photoreactive probe and a biotin-phenol subject probe and the methods disclosed herein. FIG. 9 shows total identified proteins from a nucleolar labeling experiment (FIG. 8), in which proteins are ranked by their fold-change ratio, labeled cells over unlabeled cells in logarithm.

FIG. 10 shows previously unidentified proteins in stress granules (SG) identified using the probes and methods disclosed herein. The proteome composition reveals the accuracy and the capability to discover novel proteins using these methods.

FIGS. 11A-11D shows strong photolabeling using photoreactive moieties. FIGS. 11A-11C shows chemical structures of the ruthenium, riboflavin, and Rose Bengal moieties.

FIG. 11D shows fluorescent labeling of cells using the photoreactive moieties shown in FIGS. 11A-11C.

FIG. 12 shows successful conjugation of photoreactive moieties with antibody.

FIGS. 13A-13B shows results of successful conjugation of photoreactive moieties with antibody.

FIGS. 14A-14C show results from specific photolabeling of induced cells using conjugation of a ruthenium based photoreactive moieties with antibody. FIG. 14A shows illumination at 470 nm. FIG. 14B shows a control (no light). FIG. 14C shows a Z-axis (side view) of the cells shown in FIG. 14A.

FIG. 15 shows selective photolabeling of mouse brain tissue samples using a probe of ruthenium based photoreactive moieties with antibody. Control (no light) shows no labeling.

FIG. 16 shows experimental results showing photolabeling of mouse brain tissue samples using a probe of ruthenium based photoreactive moieties with antibody and neutravidin staining.

FIG. 17 shows experimental results showing detection of nucleoli regions using a probe of ruthenium based photoreactive moieties with and a secondary antibody bait. Photolabeling signals were labeled using two-photon technique.

FIGS. 18A-18C show experimental results showing detection of photolabeled subcellular compartments using probe with ruthenium based photoreactive moieties and an Alexa fluor 568 secondary antibody bait to bind primary antibodies. FIG. 18A shows nucleoli detection using a rabbit anti-nucleolin antibody. FIG. 18B shows experimental results showing nuclear pore complex detection using a mouse anti-NPC antibody. FIG. 18C shows stress granule detection using a mouse anti-Ras GTPase-activating protein binding protein 1 antibody.

FIG. 19 shows experimental results showing specific 2 photon labeling on fixed mouse brain tissue.

FIG. 20 shows experimental results showing photolabeled stress granules using horseradish peroxidase activated desthiobiotin to covalently bind to tyrosine residues on and proximal to the enzyme site.

FIG. 21 shows proximity labeling of photolabeled mouse liver tissue samples using horseradish peroxidase activated desthiobiotin to covalently bind to tyrosine residues on and proximal to the enzyme site.

FIG. 22 shows an example of a workflow using a photoactive kit coupled with a microscopic photolabeling system followed by mass spectrometry analysis as described herein.

FIG. 23 shows experimental results showing confocal micrographs depicting precise and accurate photolabeled (PL) stress granules by antibody-ruthenium photoactive probe using a two-photon labeling system at wavelength=780 nm.

FIG. 24A-24C shows of two photon labeling using an antibody-ruthenium photoactive probe. FIG. 24A shows a Venn diagram of three biological replicates of two photon labeling Ab-Ru photoactive probe. FIG. 24B shows Volcano plot of relative protein levels in photolabeled samples to control samples (PL/CTL ratio) in a LOG2 scale. Over-represented (enriched) proteins are shown in the upper right bounded to arrow a and arrow b. FIG. 24C shows that 74% of true positive rate (annotated as stress granules (sg) are found in the top 50 proteins ranked by PL/CTL ratio.

FIGS. 25A-25B show results illustrating validation of potential stress granules proteins by immune-fluorescent detection. FIG. 25A shows that 37 proteins in the top 50 proteins ranked by PL/CTL ratio were annotated as stress granule proteins. FIG. 25B shows a proteome composition analysis confirming accuracy using the methods herein to detect stress granule proteins and the capability to discover novel stress granule biomarkers using these methods.

### DETAILED DESCRIPTION

Described herein are systems, kits, and methods useful for identifying, tagging, obtaining, and analyzing biomolecules and their neighboring biomolecules. The kits and methods may be particularly useful for analyzing biomolecule interactions in biological samples, such as analyzing proteins, nucleic acids, carbohydrates, or lipids in cell or tissue samples. The kits and methods may be advantageously useful for identifying and/or isolating previously unknown biomarkers (e.g., proximal or neighboring molecules), such as using protein sequencing and/or mass spectrometry analysis. The kits and methods utilize photoreactive materials that can label biomolecules and their neighboring biomolecules. The photoreactive kits described herein may be particularly useful for specifically labeling subsets of biomolecules in subcellular regions of cells using an image guided microscope with precision illumination control such as the system described in U.S. Patent Publication No. 2018/0367717, to enable automatic labeling of cellular biomolecules of interest. The kits can be used for in situ tagging of biomolecules such as proteins inside cells or tissues and that can be followed by proximity labeling such as using Tyramide Signal Amplification (TSA). The biomolecules can be further analyzed by analytical techniques such as mass spectrometry and sequencing. These kits may be especially useful for performing omics studies, such as genomics, proteomics, and transcriptomics, and for finding relevant biomarkers for diagnosis and treatment.

### Abbreviations and Definitions

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Amino acids described herein may be conservatively substituted so long as conservatively substituted peptide enables the desired function (such as recognition by a protease). Examples of conservative substitutions include Thr, Gly, or Asn for Ser and His, Lys, Glu, Gln for Arg. Conservative substitutions are described in e.g., Molecular Cloning: A Laboratory Manual, Fourth Edition, Green and Sambrook, eds., Cold Spring Harbor Laboratory Press, Cold Spring Harbor 2014, as well as corrections and updates thereto.)

The term “antibody” refers to immunoglobulin and related molecules and includes monoclonal antibodies, polyclonal antibodies, monomers, dimers, multimers, multispecific antibodies (e.g., bispecific antibodies), heavy chain only antibodies, three chain antibodies, single chain Fv, nanobodies. An antibody may be a polyclonal or monoclonal or recombinant antibody. Antibodies may be murine, human, donkey, goat, humanized, chimeric, or derived from other species. As used herein, when an antibody or other entity “specifically recognizes” or “specifically binds” an antigen or epitope, it preferentially recognizes the antigen in a complex mixture of proteins and/or macromolecules and binds the antigen or epitope with affinity, which is substantially higher than to other entities not displaying the antigen or epitope. A primary antibody binds specific antigen. A secondary (tertiary, etc.) antibody binds specifically to another antibody and typically to a class or subclass of antibodies, usually through the Fc domain on the other antibody.

The term “antigen binding fragment” refers to a fragment of an antibody that binds to an antigen or epitope.

The term “bait molecule” refers to a molecule that specifically interacts with a molecule of interest, which may be referred to as a target (or prey). Examples of bait molecules include an antibody, CLIP-tag, a drug, a nucleic acid, a fluorescent in situ hybridization (FISH) probe, protein A, protein G, protein L, protein A/G, protein A/G/L, another small molecule, and a SNAP-tag.

The term “binding” refers to a first moiety physically interacting with a second moiety, wherein the first and second moieties are in physical contact with one another.

The term “biotin derivative” refers to a biotin moiety, including biotin and variations of biotin, such as biotin with an open ring or substitutions. Typically, a biotin derivative is easily detectable with a biotin-binding entity or protein, such as avidin, NeutrAvidin, or streptavidin.

The term “biotin binding proteins” refers to proteins that specifically bind biotin with high affinity. Examples of biotin detection reagents are structural analogues avidin, NeutrAvidin, or streptavidin that can each bind four biotins per biotin binding protein molecule.

The term “catalyzed reporter deposition” (CARD) refers an enzyme catalyzed deposition of a detectable molecule on or near target biomolecules (e.g., carbohydrates, lipids, nucleic acids, or proteins). In some embodiments, the enzyme in an enzyme catalyzed deposition is horseradish peroxidase (HRP) and the detectable molecule is tyramide or digoxygenin (DIG).

The term “click chemistry” refers to a chemical approach that easily joins molecular building blocks. Typically, click chemistry reactions are efficient, high-yielding, reliable, create few or no byproducts, and are compatible with an aqueous environment or without an added solvent. An example of click chemistry is cycloaddition, such as the copper (I)-catalyzed [3+2]-Huisgen 1,3-dipolar cycloaddition of an alkyne and azide leading to the formation of 1,2,3-triazole or Diels-Adler reaction. Click chemistry also includes copper free reactions, such as a variant using substituted cyclooctyne (see e.g., J. M. Baskin et al., Proc. Natl. Acad. Sci. U.S.A. 2007 Oct. 23, 104 (43), 16793-16797.) Other examples of click chemistry are nucleophilic substitutions; additions to C—C multiple bonds (e.g., Michael addition, epoxidation, dihydroxylation, aziridination); and nonaldol like chemistry (e.g., N-hydroxysuccinimide active ester couplings). Click chemistry reactions can be bioorthogonal reactions, but do not need to be.

The term “conjugate” refers to a process by which two or more molecules specifically interact. In some embodiments, a tag and a label conjugate. In some embodiments, a bait and a biomolecule conjugate.

The term “conjugatable” refers to a molecule that can specifically come together with another molecule to which it can be conjugated. In some embodiments, a bait is conjugatable to a biomolecule of interest. In some embodiments, a connector is conjugatable to a primary subject probe.

The term “detectable label” refers to a compound or composition which is or is configured to be conjugated directly or indirectly to a molecule. The label itself may be detectable and be a directly detectable label (such as, e.g., fluorescent labels such as fluorescent chemical adducts, radioisotope labels, etc.), or the label can be indirectly detectable (such as, e.g., in the case of an enzymatic detectable label, the enzyme may catalyze a chemical alteration of a substrate compound or composition and the product of the reaction is detectable). Examples of detectable labels include e.g., a biotin label, a fluorescent label, horseradish peroxidase, an immunologically detectable label (e.g., a hemagglutinin (HA) tag, a poly-histidine tag), another light emitting label, and a radioactive label. An example of an indirect label is biotin, which can be detected using a streptavidin detection method.

The term “immunoglobulin-binding peptides” refers to peptides that are capable of specifically binding with high affinity to regions of an immunoglobulin molecule (antibody) other than the complementarity determining regions (CDR)/fragment antigen binding (Fab) regions. Immunoglobulin-binding peptides are other than antibodies that bind to other antibodies (e.g., other than secondary antibodies). Immunoglobulin-binding peptides typically bind to the Fc (fragment, crystallizable) region of immunoglobulins (antibodies). Immunoglobulin-binding peptides are typically immunoglobulin-binding proteins, mimics, and variations thereof, including recombinant variants, of immunoglobulin-binding bacterial proteins. Examples of non-antibody immunoglobulin-binding proteins include Protein A, Protein G, Protein L, Protein Z, Protein A/G, and Protein A/G/L. Protein A and Protein G are bacterial proteins originally obtained from the bacterium Staphylococcus aureus and Group G Streptococci, respectively, and have high affinity for the Fc region of IgG type antibodies. Protein A/G combines the binding domains of protein A and protein G. Protein A/G/L combines binding domains of protein A, protein G, and protein L. Protein A, Protein G, Protein L, Protein Z, Protein A/G, and Protein A/G/L share structural similarities.

The term “instructional material” includes a publication, a recording, a diagram, a link, or any other medium of expression which can be used to communicate the usefulness of one or more compositions of the invention for its designated use. The instructional material of a kit of the invention may, for example, be affixed to a container which contains the composition or components or be shipped together with a container which contains the composition or components. Alternatively, the instructional material may be shipped separately from a container with the intention that the instructional material and a composition or component be used cooperatively by the recipient.

The term “label” refers to a molecule which produces or can be induced to produce a detectable signal. In some embodiments, a label produces a signal for detecting a neighboring biomolecule. Examples of labels that can be used include avidin labels, NeutrAvidin labels, streptavidin labels to detect a biotin tag.

The term “linker” refers to a structure which connects two or more substructures. A linker has at least one uninterrupted chain of atoms extending between the substructures. The atoms of a linker are connected by chemical bonds, typically covalent bonds.

The phrases “bound to”, “coupled to”, “conjugated to”, “conjugatable to”, “attached to” and “linked to” refer to being directly or indirectly bound/conjugated/attached/linked. For instance, the bait molecule can be directly bound to the photoreactive moiety without intervening atoms, groups, or moieties therebetween; alternatively, it may be indirectly bound to the photoreactive moiety by one or more intervening atoms, groups moieties or linkers therebetween. The intervening atoms, groups, moieties, or linkers may include, for example, one or more non-carbon atoms, groups, or moieties, or an unsubstituted or substituted alkylene or alkenylene group which may include amine, amide, ether, ester or thioester linkages, and optionally be interrupted by one or more heteroatoms and/or rings, including aromatic rings optionally substituted.

The term “mass spectrometer” refers to an instrument for measuring the mass-to-charge ratio of one or more molecules in a sample. A mass spectrometer typically includes an ion source and a mass analyzer. Examples of mass spectrometers includes matrix assisted laser desorption ionization (MALDI), continuous or pulsed electrospray (ES) ionization, ionspray, magnetic sector, thermospray, time-of-flight, and massive cluster impact mass spectrometry.

The term “mass spectrometry” refers to the use of a mass spectrometer to detect gas phase ions.

The term “mass spectrometry analysis” includes linear time-of-flight (TOF), reflectron time-of-flight, single quadruple, multiple quadruple, single magnetic sector, multiple magnetic sector, Fourier transform, ion cyclotron resonance (ICR) or ion trap.

The term “photoactivated” or “light activated” refers to excitation of atoms by means of radiant energy (e.g., by a specific wavelength or wavelength range of light, UV light, etc.). In some examples, a photoactivated catalyst promotes covalent bond formation between a tag-bearing phenol and an amino acid.

The term “peptide” refers to a polymer in which the monomers are amino acids and the monomers are joined together through amide bonds. A peptide is typically at least 2, least 5, least 10, least 20, least 50, least 100, or at least 500 or more amino acids long.

The term “photoreactive moiety” refers to a functional moiety, which, upon exposure to light (e.g., a specific wavelength or wavelength range of light, UV light, etc.) becomes activated. In some examples, a photoreactive moiety may promote covalent bond formation between a subject probe and an amino acid or a biomolecule.

The term “proximity molecule” or neighboring molecule refers to a molecule that is near another molecule (generally a molecule of interest). A proximity molecule or neighbor molecule may be bound to the molecule of interest (e.g., covalently or non-covalently) or may be close by and not bound to the molecule of interest.

The term “prey” refers to a binding partner of a bait molecule. For example, if an antibody is a bait, a corresponding protein to which the antibody can bind is the corresponding prey. In some embodiments, a bait can bind with a single prey. In some embodiments, a bait can bind with more than one prey.

The term “protein tag” refers to peptide sequences of amino acids. Protein tags can typically be conjugated to a label. An example of a protein tag is a “self-labeling” tag. Examples of self-labeling tags include BL-Tag, CLIP-tag, covalent TMP tag, HALO-tag, and SNAP-tag. SNAP-tag is a ˜20 kDa variant of the DNA repair protein 06-alkylguanine-DNA alkyltransferase that specifically recognizes and rapidly reacts with benzylguanine (BG) derivatives. During a labeling reaction, the benzyl moiety is covalently attached to the SNAP-tag, releasing guanine. CLIP-tag is a variation of SNAP-tag configured to react specifically with O2-benzylcytosine (BC) derivatives rather than benzylguanine (BG).

The term “secondary antibody” refers to an antibody that specifically recognizes a region of another antibody. A secondary antibody generally recognizes the Fc region of a particular isotype of antibody. A secondary antibody may also recognize the Fc from one or more particular species.

The term “small molecule” refers to low molecular weight molecules that include carbohydrates, drugs, enzyme inhibitors, lipids, metabolites, monosaccharides, natural products, nucleic acids, peptides, peptidomimetics, second messengers, small organic molecules, and xenobiotics. Typically, small molecules are less than (about) 1000 molecular weight or less than about 500 molecular weight. A small molecule can be a drug molecule. A drug molecule that can be used herein is a molecule originally intended for use in the diagnosis, cure, mitigation, treatment, or prevention of device.

The term “tag” refers to a functional group, compound, molecule, substituent, or the like, that can enable detection of a target or other molecule. A tag can enable a detectable biological or physiochemical signal that allows detection via any means, e.g., absorbance, chemiluminescence, colorimetry, fluorescence, luminescence, magnetic resonance, phosphorescence, radioactivity. The detectable signal provided due to the tag can be directly detectable due to a biochemical or physiochemical property of the tag moiety (e.g., a fluorophore tag) or indirectly due to the tag interaction with another compound or agent. Typically, a tag is a small functional group or small organic compound, such as biotin, desthiobiotin, etc. In some embodiments, the employed tag has a molecular weight of less than about 1,000 Da, 750 Da, 500 Da or even smaller.

The term “tagging” refers to the process of adding a tag to a functional group, compound, molecule, substituent, or the like. Typically, tagging enables detection of a target molecule.

The term “tyramide signal amplification” (TSA), refers to a catalyzed reporter deposition (CARD), an enzyme-mediated detection method that utilizes catalytic activity of an enzyme (e.g., horseradish peroxidase) to catalyze inactive tyramide to highly active tyramide. The amplification can take place in the presence of low concentrations of hydrogen peroxide (H2O2). In some examples, tyramide can be labeled with a detectable label, such as fluorophore (such as biotin or 2,4-dinitrophenol (DNP)).

The practice of the techniques described herein may employ, unless otherwise indicated, conventional techniques and descriptions of chemistry, biochemistry, cell biology, immunology, molecular biology (including cell culture, recombinant techniques, sequencing techniques), and organic chemistry technology which are explained in the literature in the field (e.g., Molecular Cloning: A Laboratory Manual, Fourth Edition, Green and Sambrook, eds., Cold Spring Harbor Laboratory Press, Cold Spring Harbor 2014, as well as corrections and updates thereto; John D. Roberts and Marjorie C. Caserio (1977) Basic Principles of Organic Chemistry, second edition. W. A. Benjamin, Inc., Menlo Park, CA.).

### Kits

Described herein are kits that may be useful for practicing the methods described herein, e.g., for analyzing, tagging, and labeling biomolecules, identifying and/or isolating one or more biomarkers (e.g., proximal or neighboring molecules), predicting a biomarker, and identifying a biomarker list. Kits may include a photoreactive probe and/or a subject probe. Kits may additional components to facilitate the particular application for which the kit is designed. Thus, for example, kits may additionally contain materials useful for detecting a sample and/or detecting a label (e.g., enzyme substrates for enzymatic labels, filter sets to detect fluorescent labels, enzymes or associated detection reagents, including reagents for performing catalyzed reporter deposition (CARD) or signal amplification (e.g., biotin binding proteins (such as avidin, Neutravidin, streptavidin), HRP, tyramide, hydrogen peroxide, etc.). Kits may also include wash solutions, such as blocking agents, detergents, salts (e.g., sodium chloride, potassium chloride, phosphate buffer saline (PBS)) for one or more steps (e.g., after sample fixation). Kits may include variations of wash solutions, such as concentrates of wash buffers configured to be diluted before use or components to use for making one or more wash solutions) and other reagents routinely used for the practice of a particular method. The kits may include fixatives and other sample preparation materials (e.g., ethanol, methanol, formalin, paraffin, etc.)

The kits can include instructional materials disclosing methods for generating or modifying one or more probes, such as methods for attaching a bait molecule to a photoreactive moiety to prepare a photoreactive probe, applying the photoreactive probe to a sample, conjugating the bait molecule of the photoreactive probe to a prey molecule (in the sample), removing (washing away) unconjugated photoreactive probe from the sample, applying a tag-bearing subject probe (also referred to herein as a primary subject probe) to the sample, photoactivating the photoreactive probe to allow the tag-bearing subject probe to bind to a molecule of interest, removing (washing away) unbound subject probe, and applying labels to the sample. Kits can also or instead include instructional materials teaching the use of the photoreactive probes, the subject probes, the labels, and wash solutions and the like.

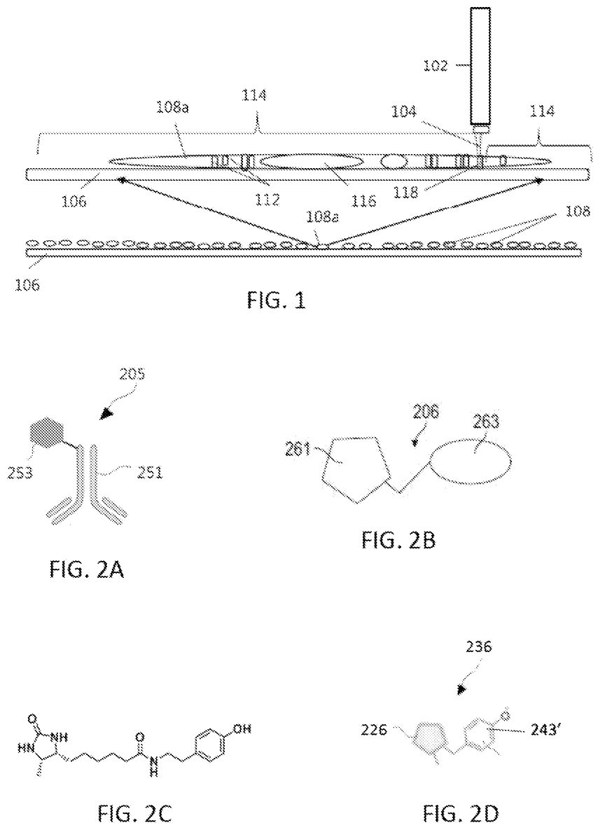

The photoreactive kits described herein can advantageously be used with a microscope system, such as the systems described herein and in U.S. Patent Publication No. 2018/0367717 A1, to enable automatic labeling of cellular biomolecules proximal to a biomolecule of interest. The labeled molecules may be directly adjacent the biomolecule of interest or may be close-by but not directly adjacent, such as when intervening molecules are present between the biomolecule of interest and cellular biomolecules for capture or analysis. Molecules that are close-by but not adjacent to a molecule of interest may be part of cell structure or otherwise contribute to a cell microenvironment of interest. FIG. 1 shows a schematic depiction of a system useful for photoselective spatial tagging and labeling of biomolecules. The bottom part of FIG. 1 shows substrate 106, such as a microscope stage, and a monolayer of a plurality of cells 108 disposed on the substrate 106. In some embodiments, the surface of an entire substrate, or a portion of the substrate, can be analyzed using an automated microscope system to identify a region of interest. For example, a sample can be stained or labeled to identify a region of interest. The top part of FIG. 1 shows an expanded view of cell 108a, one of the plurality of cells 108. The cell 108a has a nucleus 116 and a plurality of different types of organelles 112, such as cell membranes, mitochondria, ribosomes, and vacuoles. Microscope system 102 selectively shines narrow band of light 104 onto region of interest (ROI) 118 for analysis of the region of interest 118. The illumination can be in a select area, and other (and larger) regions 114 of the cell and substrate are not illuminated. As explained in more detail below, narrow band of light 104 activates a photoreactive probe to allow for bonding of a subject probe with a biological sample in only the region of interest 118.

A photoreactive probe can be represented by formula (I):

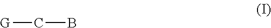

wherein the C portion includes a chemical bond or a linker, the B portion includes a photoreactive moiety bound to the C portion; and the G portion includes a bait molecule bound to the C portion and configured to conjugate with a biological sample.

In some specific examples, a photoreactive probe can have formula (IA):

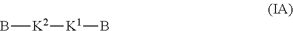

wherein a linker C of the photoreactive probe includes a K¹ portion at a proximal region of the linker; and a K² portion at a distal region of the linker, wherein the photoreactive moiety B is bound to the proximal region of the linker (K¹) and the bait molecule G is bound to the distal region of the linker (K²).

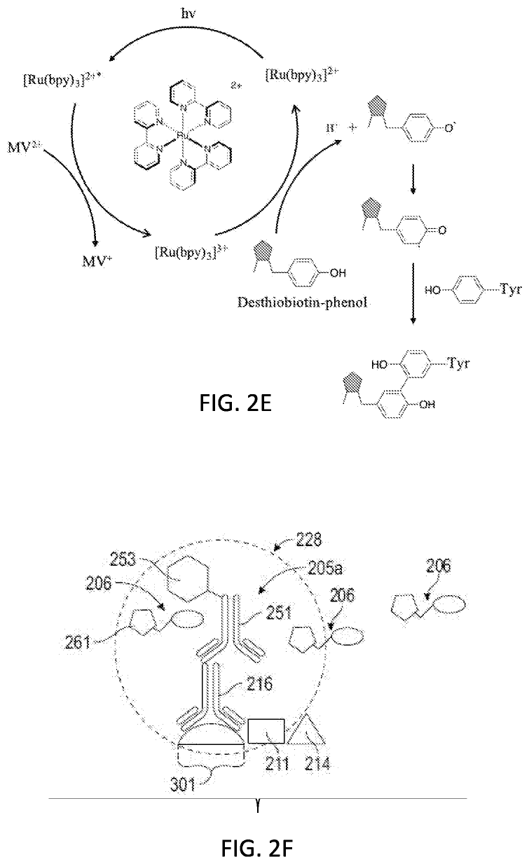

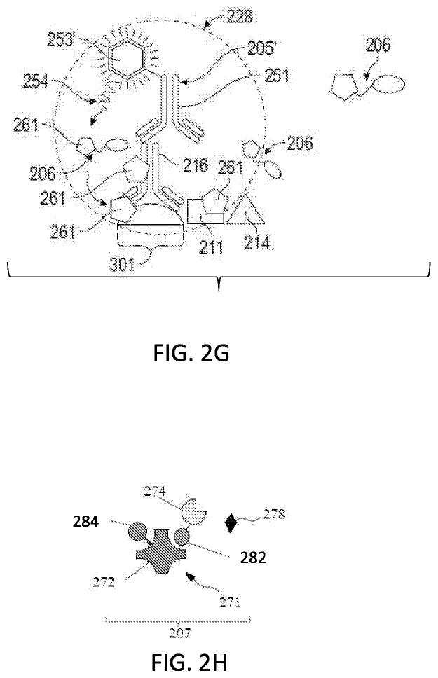

FIG. 2A schematically illustrates a photoreactive probe 205 with a bait molecule 251 conjugated with a photoreactive moiety 253. FIG. 2B schematically illustrates a subject probe 206 (sometimes also referred to herein as a primary subject probe) with a tag portion 261 and a subject moiety 263. FIG. 2C shows a subject probe with a desthiobiotin tag and a phenol subject moiety. Although FIGS. 2A and 2C (taken together) shows the bait molecule 251, the tag portion 261 and the subject moiety 263 as an antibody (such as a secondary antibody), a desthiobiotin and a phenol, respectively, any bait molecule, tag portion and subject moiety as described herein can be used. FIG. 2D schematically illustrates a subject probe 236 with biotin tag 226 and an activated phenolic group 243′. FIG. 2D illustrates the subject probe after light illumination of a photoreactive probe (probe 205), which in turn has activated a phenol group of a subject probe, such as the phenol group shown in FIG. 2C, so as to allow the activated subject probe to have phenolic group with a free radical (e.g., a reactive group). The free radical can be especially useful for conjugating with nearby proteins. Biotin tag 226 (or another tag) can later be used to isolate or enrich for a tagged neighboring protein to which it is attached. For example, when tag 226 is a biotin, a neighboring protein can be isolated or enriched for by using biotin-avidin affinity techniques (biotin-biotin binding protein affinity techniques).

FIG. 2E illustrates interactions of a ruthenium photoreactive probe and subject probe having a desthiobiotin tag and phenol subject moiety to effect labeling of a neighboring protein. After entering the reaction range of photoreactive moiety 253′/dashed line circle 228 as shown in FIG. 2G, the phenol becomes a tyrosyl radical, which will specifically react with and conjugate to amino acid tyrosine residues of nearby proteins.

FIGS. 2F-2G schematically illustrates steps in biomolecule proximity tagging in a target region 228. As can be seen in FIG. 2F, after the photoreactive probe 205 is delivered to the sample, such as a cell or organelle sample, and allowed to detect and bind to a target biomolecule 301 (e.g., through bound photoreactive probe 205a) such as a primary antibody, or a carbohydrate, lipid, nucleic acid, or protein in the sample, subject probe 206 is added to the sample. FIG. 2G illustrates photoexcitation of the photoreactive probe 205 to form photoexcited photoreactive probe 205′ with photoexcited photoreactive moiety 253. Photoexcited photoreactive probe 205′ is conjugated to the target biomolecule 301 and allows (arrow 254) subject probe 206 to bind to neighbor molecules upon photoexcitation of the photoreactive probe 205 conjugated to the target biomolecule 301. (FIG. 2G shows tag portion 261 of subject probe 206 bound to molecules, such as neighbor molecule 261 and for clarity, the rest of probe 206 (including subject moiety 263) is not shown but is present). Although this example shows a primary antibody recognizing and attached to target biomolecule of interest 301, other photoreactive probes 205 with other bait molecules 251 can detect and bind to any target as described herein. Subject probe 206 contains tag portion 261 which can be detected using the methods described herein. For example, if the tag portion 261 is a biotin derivative (including biotin), it can be detected using at least one biotin binding proteins (avidin, streptavidin, and/or neutravidin) detection method. In some examples, this photo-assisted enzymatic labeling method can advantageously significantly reduce the background noise by means of the selective photocatalystic probe (probe 205), label cytosolic organelles with higher resolution and finer detail and achieve subcellular proteomics without tyramide signal amplification.

FIG. 2H shows an example of a labeling system 207 that can be used with the subject probe 206 shown in FIG. 2B to label biomolecules neighboring a target biomolecule of interest. The labeling system 207 includes a labeling complex 271 with a connector 272 and an enzyme or catalyst 274, and an additional subject probe 278. FIG. 2H also shows fluorescent connector moiety 284. Fluorescent connector moiety 284 can be useful for identifying which area is labeled by subject probe 206, when tag portion 261 of subject probe 206 has a high affinity with the connector. Although this example shows a fluorescent connector moiety 284, a connector either with or without a fluorophore can be used. In some embodiments, connector 272 can be a biotin binding protein-dye conjugate (avidin-dye conjugate, streptavidin-dye conjugate, neutravidin-dye conjugate) or the like, and the enzyme or catalyst 274 can be tag-peroxidase and utilize peroxide (not shown) for activity. FIG. 2H also shows tag 282 such as biotin and can used to conjugate enzyme or catalyst 274 with the connector 272 (e.g., biotin binding protein such as avidin). After the tag (biotin) 282-enzyme 274 complex is conjugated with connector 272, which in turn is conjugated on the protein labeled by subject probe 206, enzyme or catalyst 274 can be used to catalyze a proximal reaction, which allows an additional subject probe to be activated so as to label proteins, which are near the biotin 282-enzyme 274 complex. In this case, subject probe 206 and the additional subject probe 278 could be the same or different molecules.

FIG. 2H shows the connector 272 and tag portion 261 (FIG. 2B) recognize one another and conjugate together. The enzyme or catalyst 274 can activate additional subject probe 278 and, once activated, the activated subject probe 278 (e.g., a tyramide probe) can bind to and detectably label biomolecules in its vicinity. The additional subject probe 278 can include a tag portion and a subject moiety and may be the same as or different from the subject probe 206. Any tag portion and/or subject moiety described herein for the primary subject probe 206 also can be used in the additional subject probe 278.

FIG. 2I shows a comparison of direct photochemical labeling (top, labeled Process II) and photo-assisted enzymatic labeling (bottom, labeled Process III) using the photoreactive kits and systems described herein on a specimen with biomolecules (I) to label biomolecules in small region of interest (ROI). Prior to performing either Process II or Process III, a sample (e.g., a cell or tissue sample) containing a biomolecule of interest 210 (protein will be used herein by way of example, but other biomolecules could instead be analyzed) is analyzed and a region of interest identified. The sample can be pretreated, such as fixed and stained. For example, a sample can be fixed and stained with a cell stain (e.g., hematoxylin and eosin (H &E); Masson's trichrome stain), identified with an immunofluorescent labeled antibody recognizing a protein of interest or by other methods. Once a region of interest is identified, a complex of neighboring biomolecules within the region of interest can be analyzed using the methods and systems described herein. As illustrated in Process II in FIG. 2I, the sample can be treated with a direct photoreactive probe 212 and light directed to a region of interest in the sample (also referred to as patterned light) can activate direct photoreactive probe 212 to form activated direct photoreactive probe 212′. The activated direct photoreactive probe 212′ is able to form complexes with other molecules with a close vicinity (show by the dotted circle in Process II). The activated direct photoreactive probe 212′ can diffuse and label neighbor molecules 211 near the molecule of interest 210. However, the labeling diameter (300-600 nm) of photoactivation of the direct photoreactive probes is spatially restricted by the diffraction limit of the light sources used. Additionally, since the direct photoreactive probe is free to diffuse, any proteins in the pathway of the direct photoreactive probe (e.g., in the pathway of patterned light) can be labeled. Process II also shows it labels more distant biomolecules 231. The region labeled by the activated direct photoreactive probe 212′, or labeled precision, covers a region of about 300-600 nm. This region can include biomolecules that are not in close proximity to protein of interest, and in some cases might lead to confusing, misleading, or unhelpful results.

In contrast, in Process III, shown on the bottom of FIG. 2I, photoreactive moiety 253 is pre-conjugated with bait molecule 251, forming photoreactive probe 205 (see FIG. 2A) The photoreactive moiety 253 can be pre-conjugated with bait molecule 251, such as through covalent bonds with or without a linker, such as a linker shown in FIG. 4. As illustrated in Process III in FIG. 2I, photoreactive probe 205 is delivered to the sample (specimen) on substrate 209 and bait molecule 251 (as part of photoreactive probe 205) recognizes a corresponding biomolecule of interest (e.g., target or prey). As illustrated in Step (i) in FIG. 2I, patterned light is also directed to the sample in a selected location (region of interest). However, here, the patterned light activates the photoreactive moiety 253 of the photoreactive probe 205′ now attached to the biomolecule of interest (e.g., target or prey), and the activated photoreactive probe 205′ can promote the subject moiety 263 of the primary subject probe 206 to form a covalent bond with nearby molecules or moieties in the specimen. In addition to the patterned light directing only a limited region of photoactivation, the readily accessible subject probe 206 is only activated within a restricted catalyst radius (e.g., within the reaction range of the photoreactive moiety, such as shown by target region 228) and is sufficiently reactive that it cannot undergo long-range diffusion after activation and thus primary subject probe 206 becomes covalently bound to the neighbor molecules 211 and 214. Step (i) also shows how background or unwanted labeling can be reduced using the probes and methods described herein. In Step (i), a photoreactive probe 205a is attached to a biomolecule; however, since the photoreactive probe 205 is outside the light delivery region (patterned light region), the photoreactive probe 205a is not activated, and the subject probe 206 and molecules outside the light delivery region do not bond.

Unbound subject probes are washed away with wash solution. Steps (ii) and (iii) in FIG. 2I show labeling of the molecules near the molecule of interest 210 using the labeling system 207 shown in FIG. 2H. Other labeling systems can also be used. By way of example, connector 272 of the labeling system 271 conjugates with tag portion 261 of 206 labeled biomolecule (211), the enzyme or catalyst 274 activates additional subject probe 278, forming activated subject probe 278′. Activated additional subject probe 278′ binds to neighbor molecule 211 (see additional subject probe 278″ in FIG. 2I). Since the photoreactive probe 205′ was attached to molecule of interest 210, and the subject probe 206 and the additional subject probe 278 did not diffuse very far before reacting, neighbor molecule 214 is labeled, while the more distant molecule 231 is not.

By photoselectively localizing an enzyme or catalyst 274 (e.g., as part of a labeling system 207), such as peroxidase, near a molecule of interest and labeling the neighbor molecules 214 in the region of interest using the tagging and labeling just described, the 271 catalyzed reaction describe in FIG. 2I can be localized to a region as small as <100 nm. In some variations, a larger region (e.g., up to about 200 nm, up to about 300 nm, up to about 400 nm, up to about 500 nm, up to about 1 μm, up to about 2 μm, up to about 5 μm) could be labeled. Furthermore, some molecules of interest in a sample can have multiple small regions of localization and hence a photoreactive probe can interact with different molecular complexes containing the same molecules of interest in different locations simultaneously. The photo-labeling can be used successively in more than one location. For example, after applying light as shown in FIG. 2I Process III, the light can be selectively applied to a second (third, fourth, etc.) location in the sample and this process can be repeated as many times as desired. In addition to labeling (depositing labels to) a relatively small number of neighbor molecules in a very small area of a sample, such as due to the use of the microscope analysis to direct the light and the probes described herein, and as explained below, the process can also be performed with sufficiently mild or gentle treatments so that the cell architecture remains intact during use of the methods herein, advantageously allowing detection of naturally occurring biomolecule interactions.

Non-limiting examples of bait molecules that can be used herein can include one or more of an antibody, a CLIP-tag, HaloTag, a SNAP-tag, a functional protein (e.g. protein A, protein G, protein L, protein A/G, protein A/G/L, or a protein drug), immunoglobulin binding peptides, a biotin binding protein (including avidin, streptavidin, and/or neutravidin), an RNA molecule, a small molecule (e.g. erlotinib), a nucleic acid molecule, a fluorescent in situ hybridization (FISH) probe, fragment antigen binding region, nanobody, a biologic drug, and the like. Examples of biologic drugs that can be used as bait include abatacept (Orencia); abciximab (ReoPro); abobotulinumtoxinA (Dysport); adalimumab (Humira); adalimumab-atto (Amjevita); ado-trastuzumab emtansine (Kadcyla); aflibercept (Eylea); agalsidase beta (Fabrazyme); albiglutide (Tanzeum); aldesleukin (Proleukin); alemtuzumab (Campath, Lemtrada); alglucosidase alfa (Myozyme, Lumizyme); alirocumab (Praluent); alteplase, cathflo activase (Activase); anakinra (Kineret); asfotase alfa (Strensiq); asparaginase (Elspar); asparaginase Erwinia chrysanthemi (Erwinaze); atezolizumab (Tecentriq); basiliximab (Simulect); becaplermin (Regranex); belatacept (Nulojix); belimumab (Benlysta); bevacizumab (Avastin); bezlotoxumab (Zinplava); blinatumomab (Blincyto); brentuximab vedotin (Adcetris); canakinumab (Ilaris); capromab pendetide (ProstaScint); certolizumab pegol (Cimzia); cetuximab (Erbitux); collagenase (Santyl); collagenase Clostridium histolyticum (Xiaflex); daclizumab (Zenapax); daclizumab (Zinbryta); daratumumab (Darzalex); darbepoetin alfa (Aranesp); denileukin diftitox (Ontak); denosumab (Prolia, Xgeva); dinutuximab (Unituxin); dornase alfa (Pulmozyme); dulaglutide (Trulicity); ecallantide (Kalbitor); eculizumab (Soliris); elosulfase alfa (Vimizim); elotuzumab (Empliciti); epoetin alfa (Epogen/Procrit); etanercept (Enbrel); etanercept-szzs (Erelzi); evolocumab (Repatha); filgrastim (Neupogen); filgrastim-sndz (Zarxio); follitropin alpha (Gonal f); galsulfase (Naglazyme); glucarpidase (Voraxaze); golimumab (Simponi); golimumab injection (Simponi Aria); ibritumomab tiuxetan (Zevalin); idarucizumab (Praxbind); idursulfase (Elaprase); incobotulinumtoxinA (Xeomin); infliximab (Remicade); infliximab-dyyb (Inflectra); interferon alfa-2b (Intron A); interferon alfa-n3 (Alferon N Injection); interferon beta-1a (Avonex, Rebif); interferon beta-1b (Betaseron, Extavia); interferon gamma-1b (Actimmune); ipilimumab (Yervoy); ixekizumab (Taltz); laronidase (Aldurazyme); mepolizumab (Nucala); methoxy polyethylene glycol-epoetin beta (Mircera); metreleptin (Myalept); natalizumab (Tysabri); necitumumab (Portrazza); nivolumab (Opdivo); obiltoxaximab (Anthim); obinutuzumab (Gazyva); ocriplasmin (Jetrea); ofatumumab (Arzerra); olaratumab (Lartruvo); omalizumab (Xolair); onabotulinumtoxinA (Botox); oprelvekin (Neumega); palifermin (Kepivance); palivizumab (Synagis); panitumumab (Vectibix); parathyroid hormone (Natpara); pegaspargase (Oncaspar); pegfilgrastim (Neulasta); peginterferon alfa-2a (Pegasys); peginterferon alfa-2b (PegIntron, Sylatron); peginterferon beta-1a (Plegridy); pegloticase (Krystexxa); pembrolizumab (Keytruda); pertuzumab (Perjeta); ramucirumab (Cyramza); ranibizumab (Lucentis); rasburicase (Elitek); raxibacumabreslizumab (Cinqair); reteplase (Retavase); rilonacept (Arcalyst); rimabotulinumtoxinB (Myobloc); rituximab (Rituxan); romiplostim (Nplate); sargramostim (Leukine); sebelipase alfa (Kanuma); secukinumab (Cosentyx); siltuximab (Sylvant); tbo-filgrastim (Granix); tenecteplase (TNKase); tocilizumab (Actemra); trastuzumab (Herceptin); ustekinumab (Stelara); vedolizumab (Entyvio); ziv-aflibercept (Zaltrap).

Non-limiting examples of photoreactive moieties include aryl azide, benzophenone, riboflavin, flavin, lumiflavin and or a derivative thereof, fluorescein or a derivative thereof, KillerRed (photosensitizer protein), miniSOG (photosensitizer protein), another photosensitized protein (e.g., configured to generate reactive oxygen species (ROS) upon light radiation), methylene blue or a derivative thereof, phenol, Pterin derivatives, derivatives, ruthenium-based photocatalysts, and Rose Bengal derivatives. Fluorescein is a fluorescent organic dye with four negatively charged carboxylate groups, and derivatives thereof are configured to generally preserve the active center and may have, for example, additions such hydrocarbon tails, isothiocyanate, carboxylic acid, or amine. Derivatives of other molecules (unless otherwise specified or clear from the context) generally preserve characteristic functionality of the molecule, but may otherwise be modified. In some embodiments, a plurality of photoreactive moieties is bound to an antibody (or other bait molecule) to form a photoreactive probe. The number of the photoreactive moieties bound to an antibody (or other bait molecules) may range from 1 to 50 (e.g., 1, 1-5, 1-10, 1-20, etc.).

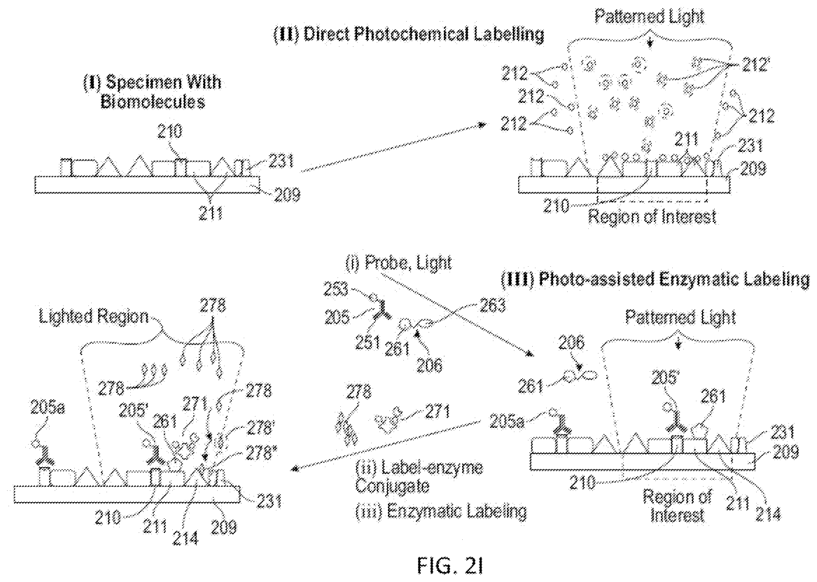

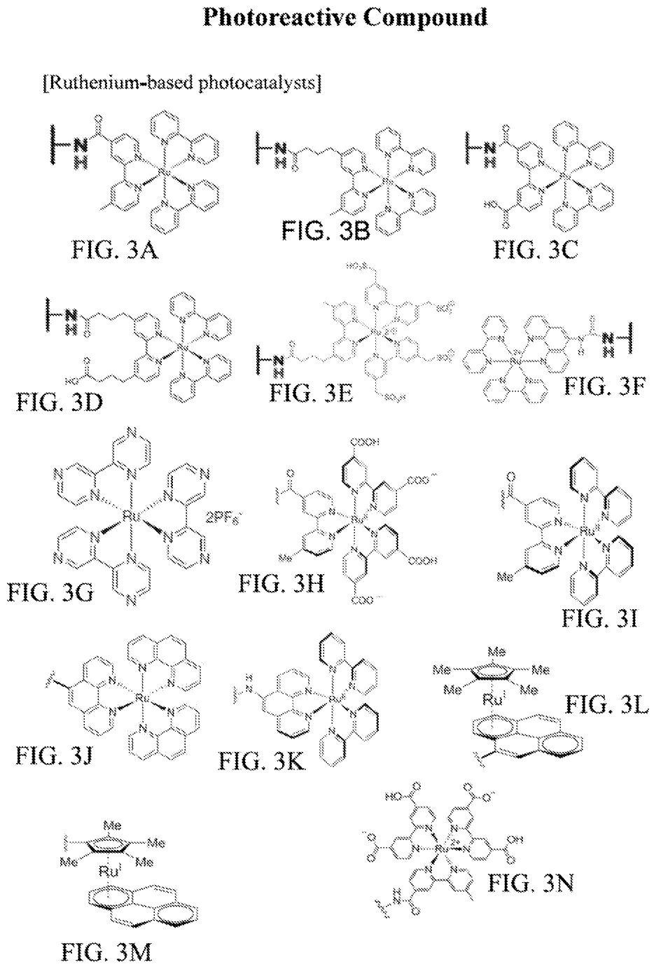

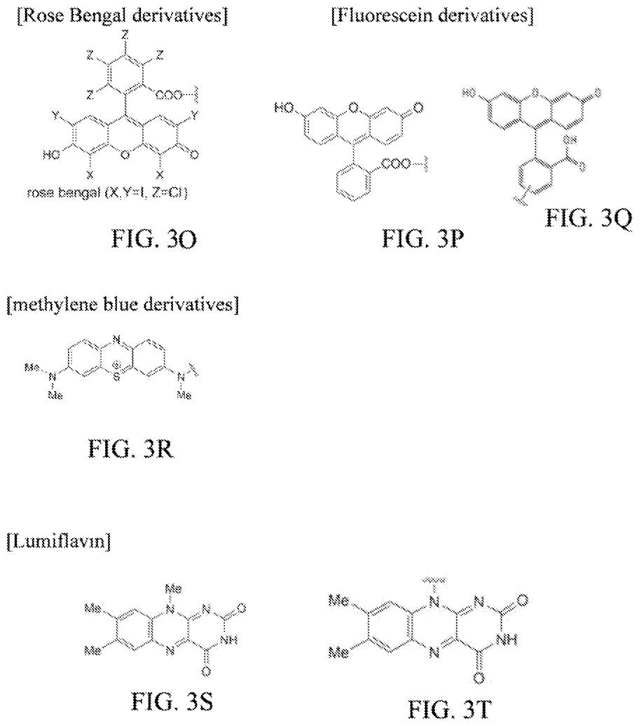

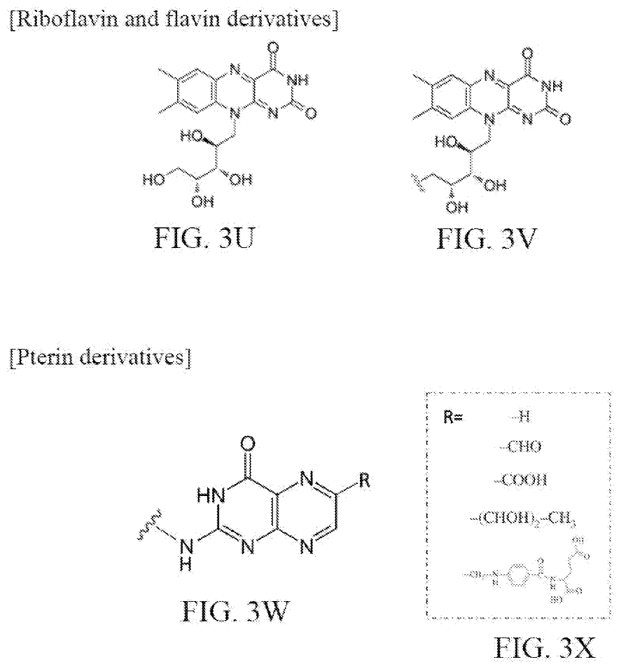

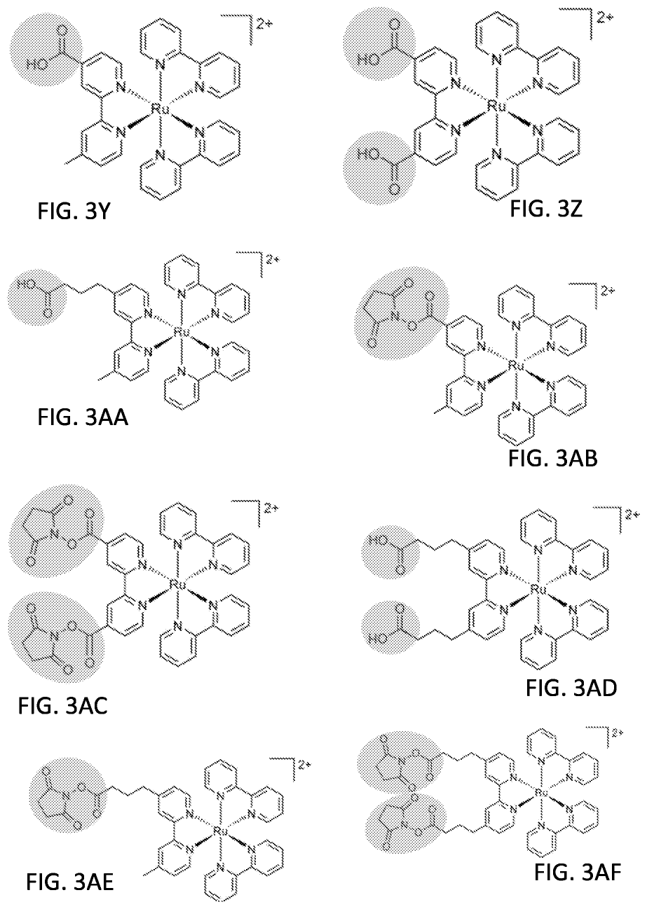

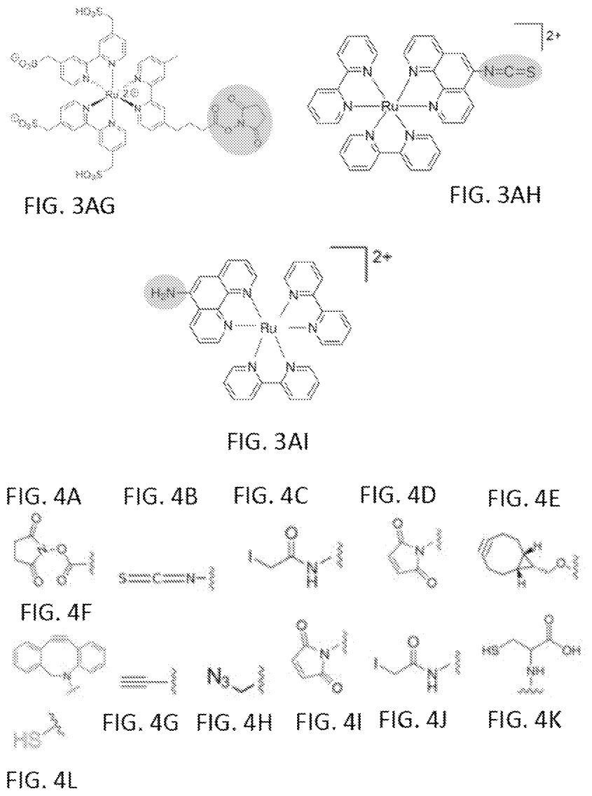

FIGS. 3A-3G and 3Y-3AI show examples of photoreactive moieties (e.g., 253 photoreactive moiety) that can be used in the photoreactive probes (e.g., photoreactive probe 205) described herein. FIGS. 3A-3N shows ruthenium-based photocatalysts. In FIGS. 3Y-3AI, the shaded area shows the location of linker (covalent bond) attachment. FIG. 3O shows Rose Bengal derivatives. FIGS. 3P-Q shows fluorescein derivatives. FIG. 3R shows methylene blue derivatives. FIGS. 3S-T shows Lumiflavin derivatives. FIGS. 3U-3V shows Riboflavin and flavin derivatives. FIGS. 3W-3X shows Pterin derivatives. The selection of a particular photoreactive moiety can depend on the desired wavelength and the types of the bait molecule. For example, the constituents of the photoreactive probe and constituents for the pre-probe analysis can be chosen so as to not interfere (or minimally interfere) with each other.

In some embodiments, the ruthenium-based compound represented by formula (II) may be used for the B portion (photoreactive moiety) of the photoreactive probe represented by formula (I) or (IA):

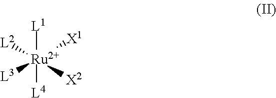

wherein, L1, L2, L3, and L4 are each independently a ligand; and X¹ and X² are each independently a ligand having a reactive moiety, wherein the reactive moiety can be bound to the C portion of the formula (I) as listed above. For instance, X¹ and X² each may independently be 3-ethynylpyridine, 3-(bromomethyl)pyridine, maleimide, 4′-methyl-4-carboxybipyridine-N-succinimidyl ester, nicotinaldehyde, 1-(4-(pyridin-3-yl)-1H-1,2,3-triazol-1-yl) ethanone, 4-pentynenitrile, 4-aminobutyne, or the like; and L¹ and L² may be joined to form a first bidentate ligand and L³ and L⁴ may be joined to form a second bidentate ligand, wherein the first bidentate ligand and the second bidentate ligand each may independently be 2,2′-bipyridyl (bpy), 4′-methyl-4-carboxybipyridine-N-succinimidyl ester, 4,4′-dicyano-5,5′-dimethyl-2,2′-bipyridine (CN-Me-bpy), 4,4′-dimethyl-2,2′-bipyridine (dmb), 4,4′-di-tert-butyl-2,2′-bipyridine (dbpy), 4,4′,5,5′-tetramethyl-2,2′-bipyridine (tmb), 2-phenylpyridine (ppy), 6-bromo-2,2′-bipyridine, 6,6′-dibromo-2,2′-bipyridine, 5-bromo-2,2′-bipyridine, 6-amino-2,2′-bipyridine, 6,6′-diamino-2,2′-bipyridine, 2,2′-bipyridine-6-carbonitrile, 2,2′-bipyridine-6,6′-bis(carbonitrile), 2,2′-bipyridine-6-carboxylic acid, 2,2′-bipyridine-6,6′-dicarboxylic acid, biquinoline, or the like. Accordingly, the ruthenium-based compound of formula (II) may be [Ru(bpy)₂(methyl-bpy-NHS)]²⁺, [Ru(bpy)₂(bpy-(NHS) 2)]²⁺, [Ru(bpy)₂ (methyl-bpy-(CH2)3-NHS)]²⁺, [Ru(bpy)₂(bpy-((CH₂)₃—NHS))₂]²⁺, Sulfo-TAG NHS-Ester, [Ru(bpy)₂(methyl-bpy-COOH)]²⁺, [Ru(bpy)₂(bpy-(COOH)₂)]²⁺, [Ru(bpy)₂ (methyl-bpy-(CH₂)₃COOH)]²⁺, [Ru(bpy)₂(bpy-((CH2)₃COOH))₂]²⁺, a salt (e.g., bis(hexafluorophosphate)) thereof, or any combinations thereof. The NHS-ester group may be replaced with a chemical group, such as a maleimide group, an iodoacetyl group, a cysteine/thiol group, a click chemical group or the like. The carboxyl group may be replaced with a chemical group, such as maleimide group, an iodoacetyl group, a cysteine/thiol group, a click chemical group or the like. The click chemical group may be alkyne, BCN, DBCO, N₃, or the like. In certain embodiments, the ruthenium-based compound of formula (II) is selected from the group consisting of [Ru(bpy)₂(isothiocyanato-phenanthroline)]²⁺, [Ru(bpy)₂(aminophen anthroline)]²⁺, [Ru(bipyridine)₂(3-ethynyl-pyridine)₂]+, Ru(bipyridine)₂ (3-ethynylpyridine)₂Cl₂, Ru(bipyridine)₂(3-ethynylpyridine)₂(PF₆)₂, [Ru(biquinoline)₂(4-pentynenitrile)₂]+, Ru(biquinoline)₂(4-pentyne nitrile)₂Cl₂, Ru(biquinoline)₂(4-pentynenitrile)₂(PF₆)₂, [Ru(bipyridine)₂(4-aminobutyne)₂]⁺, [Ru(bipyridine)₂(4-pentynenitrile)₂]⁺, [Ru(bipyridine)² (nicotinaldehyde)₂]+, [Ru(bipyridine)2(1-(4-(pyridin-3-yl)-1H-1,2,3-triazol-1-yl) ethanone)₂]⁺, [Ru(bipyridine)₂(3-(bromo methyl)pyridine)₂]⁺, [Ru(bipyridine)2(maleimide)₂]⁺, Ru (4,4-dicarboxylic acid-2,2′-bipyridine)(4,4′-bis(p-hexyloxystyryl)-2,2-bipyridine)(NCS)₂, cis-Bis(isothiocyanato) bis(2,2′-bipyridyl-4,4′-dicarboxylato)ruthenium(II), cis-Bis(2,2′-bipyridine)dichloro ruthenium(II) hydrate, Bis(4,4-dicarboxy-2,2-bipyridine)dichloro ruthenium(II), Di-tetrabutylammonium cis-bis (isothiocyanato)bis (2,2′-bipyridyl-4,4′-dicarboxylato)ruthenium(II), a salt thereof, a stereoisomer thereof, or tautomer thereof and any combinations thereof.

FIGS. 4A-4L shows exemplary moieties for the C (linker) portion in the photoreactive probe represented by formula (I) listed above. For the C portion of the photoreactive probe, some embodiments can use, for example, NHS—BCN, NHS-(PEG)ₙ-BCN, NHS-DBCO, NHS-(PEG)ₙ-DBCO, NHS-alkyne, NHS-(PEG)ₙ-alkyne, NHS—N₃, NHS-(PEG)ₙ-N₃, NHS-maleimide, NHS-(PEG)ₙ-maleimide, NHS-iodoacetyl group, NHS-(PEG)ₙ-iodoacetyl group, NHS-cysteine/thiol group, NHS-(PEG)ₙ-cysteine/thiol group, maleimide-peptide/amino acid, maleimide-(PEG)ₙ-peptide/amino acid, maleimide-oligonucleotide, iodoacetyl-peptide/amino acid, iodoacetyl-(PEG)ₙ-peptide/amino acid, iodoacetyl-oligonucleotide or the like as the linker, wherein each n can independently be an integer of 1-20. In some embodiments, the linker includes the moiety of (PEG) n, peptide, amino acid, or oligonucleotide, and wherein each n can independently be an integer of 1-20. Other examples of polymeric linkers include other polyethylene glycols (PEG), polypropylene glycol, polyethylene, polypropylene, polyamides, and polyesters. Linkers can be linear molecules in a chain of at least one or two atoms and can include more.

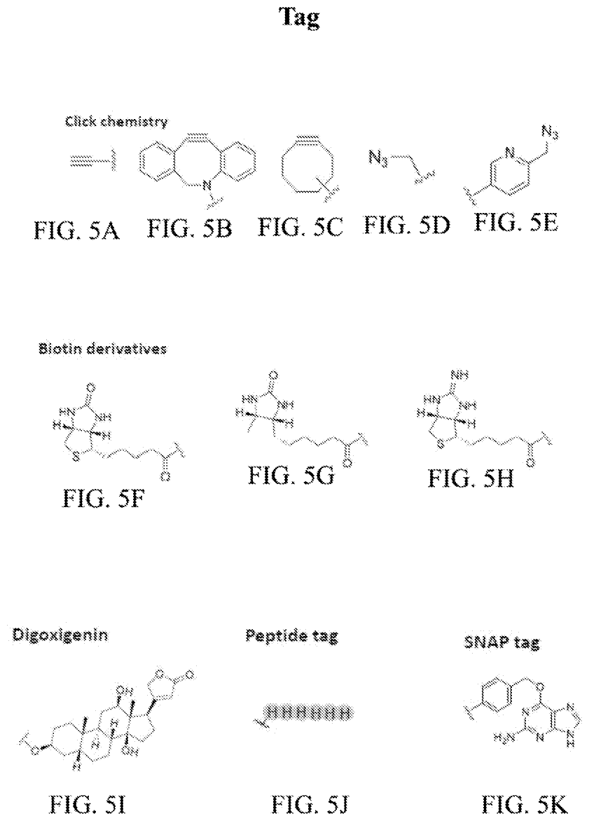

FIGS. 5A-5K show examples of tag portions that can be used in the subject probes described herein. Tag portions (sometimes also referred to herein as “tag” or “tags”) are configured to interact with a detectable label to label biomolecules neighboring a molecule of interest (a first molecule) and to generate a detectable label. FIGS. 5A-5E shows examples of click chemistry tags that can be used with the probes. A click chemistry tag may be, for example, an azide moiety or an alkyne moiety. FIG. 5F-5H shows examples of biotin derivatives that can be used as probe tags in a subject probe. FIG. 5I shows a digoxigenin moiety tag. FIG. 5J shows a peptide tag. In particular, FIG. 5J shows a poly His tag with 6 histidines (SEQ ID NO. 1). However, a histidine tag could instead include fewer or more histidines, such as 5 histidines or 7-10 histidines or more than 10 histidines. FIG. 5K shows a SNAP-tag. In some instances, a CLIP-tag, oligonucleotide, or HaloTag could also be used.

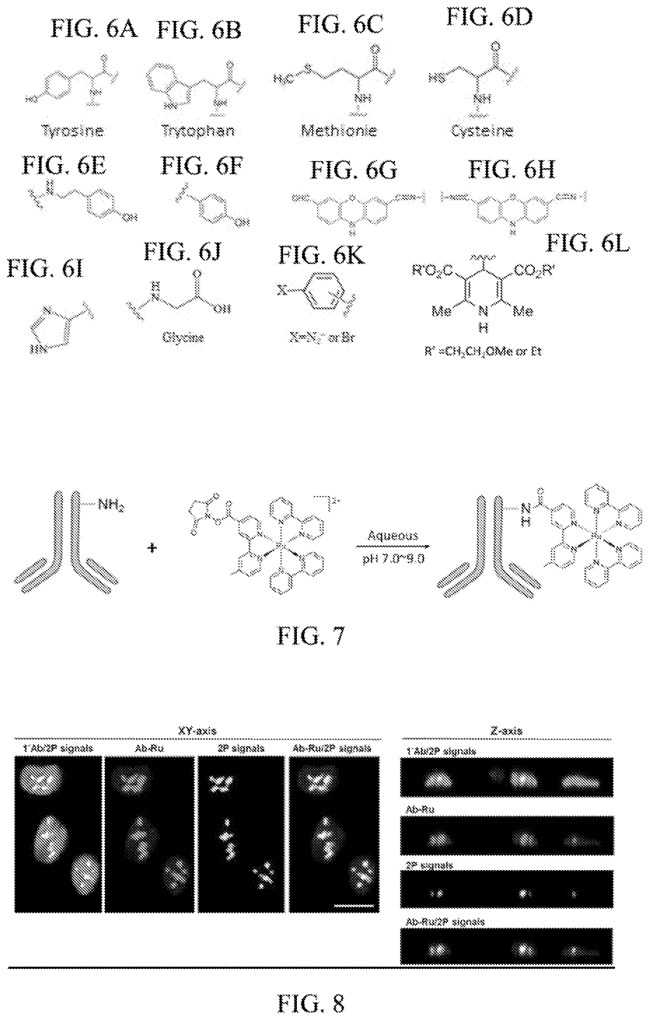

FIGS. 6A-6L shows examples of subject moieties that can be used in the subject probes described herein (e.g., along with a tag portion), such as in subject probe 206 shown in FIG. 2B. After light activation of a photoreactive probe (e.g., photoreactive probe 205), the activated photoreactive probe can generate a reactive intermediate (such as a free radical) from subject moiety 263 of a subject probe 206 and subject moiety 263 can form a covalent bond with an amino acid or other biomolecules in proximity to it (e.g., a neighboring molecule, such as neighbor molecule 211). The amino acid may be alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine or the like. In some variations, a subject moiety can form a bond with neighboring carbohydrates, lipids, or nucleic acids.

In an implementation of photocatalytic ruthenium complex-antibody conjugate as a photoreactive probe 205, bis(2,2′-bipyridine)-4′-methyl-4-carboxybipyridine-ruthenium can be specifically conjugated to a secondary antibody through NHS-amide linkage and the secondary antibody can selectively hybridize to a primary antibody of the region of interest (ROI) (see e.g., FIGS. 2E-2E.) [Ru(bpy)₃]²⁺ is a photocatalyst that can be excited by either single-photon or two-photon illumination around 425 nm and 780 nm, respectively. The resulting oxidized [Ru(bpy)₃]³⁺ can seize an electron from the phenolic hydroxyl group of biotin-phenol (see e.g., FIG. 2B) as a subject probe and generate a phenoxyl radical and proton to tyrosine residues in proximity. To achieve a higher density of labeling for omics studies, such as for proteomic profiling, HRP amplification can be utilized to further covalently bind tyramide radicals to nearby tyrosine residues of protein neighbors, followed by streptavidin-enrichment and on-bead digestion. Finally, subcellular/localized proteomic profiles can be obtained by performing quantitative proteomic analysis on the specimen, such as after removing a specimen from a microscope slide. The implementation is characterized by antibody-based ruthenium complex ([Ru(bpy)₃]²⁺)-antibody conjugate formation as a selective and photocatalystic probe for spatial and localized proteomic analysis, identification of novel proteins of cellular organelles that are unable to fractionate or isolate by conventional methods, and size and morphological distinguishable labeling via single-or two-photon illumination.

Described herein in some embodiments are photoreactive kits that include at least one of a photoreactive probe and a primary subject probe or both a photoreactive probe and a primary subject probe. In this and other embodiments, a photoreactive probe can be represented formula (I): G-C-B (I), wherein the C portion is a single chemical bond or a linker; the B portion includes a photoreactive moiety bound to the C portion; and the G portion includes a bait molecule bound to the C portion. In this and other embodiments, a primary subject probe includes a detectable tag and a subject moiety. The primary subject probe can include a detectable tag bound to a photoexcitable subject moiety. In some embodiments, a bait molecule of a photoreactive probe is configured to conjugate with a first molecule in a biological sample, and when the photoreactive probe is photoactivated and the primary subject probe is acted upon by the photoactivated probe to form a photoexcited primary subject probe, the photoexcited primary subject probe is configured to form a covalent bond with a target molecule in the biological sample. In some embodiments, a first molecule and a target molecule are different entities. In some embodiments, a first molecule and a target molecule are one and the same entity. Disclosure herein to a first molecule can also refer to or be applicable to a target molecule, unless context indicates otherwise.

Photoselective tagging and labeling as described herein can be performed in various types of samples, such as samples obtained from tissues, cells, or particles, such as from an entity (e.g., a human subject, a mouse subject, a rat subject, an insect subject, a plant, a fungi, a microorganism, a virus) or tissues samples or cell samples that are not from an organism, such as cell culture samples or artificial tissue scaffold samples (e.g., cultured laboratory cells, in vitro developed heart tissue, 3-d printed tissue, etc.). Samples for analysis using the probes, materials, and methods described herein can be living (live cells) or can be not living (e.g., fixed). A sample for tagging and labeling can include a monolayer sample, a multi-layer sample, a sample fixed to a substrate (e.g., a microscope slide), a sample not fixed to a substrate, a suspension of cells, or an extract, such as an in vitro cell extract, a reconstituted cell extract, or a synthetic extract. In some embodiments, a sample is not fixed (unfixed). Examples of probes useful for tagging live cells include those utilizing a small molecule or those sometimes referred to as self-labeling molecules (e.g., Clip-tag, Halo-tag, SNAP-tag). In some embodiments, a large number of cells can be automatically analyzed using the methods and materials described herein (e.g., at least about 1,000 cells, at least 10,000 cells, at least 100,000 cells, at least 1 million cells). In some embodiments, a smaller number of cells can be analyzed, such as no more than 1,000 cells, no more than 100 cells, or only a few cells or a single cell. In some embodiments a sample is fixed. For example, a cell or tissue sample may be fixed with e.g., acetic acid, acetone, formaldehyde (4%), formalin (10%), methanol, glutaraldehyde, or picric acid. A fixative may be a relatively strong fixative and may crosslink molecules or may be weaker and not crosslink molecules. A cell or tissue sample for analysis may be frozen, such as using dry ice or flash frozen, prior to analysis. A cell or tissue sample may be embedded in a solid material or semi-solid material such as paraffin or resin prior to analysis. In some embodiments, a cell or tissue sample for analysis may be subject to fixation followed by embedding, such as formalin fixation and paraffin embedding (FFPE). A bait portion (of a photoreactive probe) can be one or more of a nucleic acid, a protein, and a small molecule. A nucleic acid bait portion can be DNA, cDNA, or RNA. A nucleic bait portion can be an in situ hybridization probe, such as a fluorescent in situ hybridization probe or a non-fluorescent in situ hybridization probe, such as a chemiluminescent in situ hybridization probe (CISH). An in situ hybridization probe is one or more nucleic acid strands composed of DNA, cDNA, or RNA that contains or can be modified to contain a fluorescent or other detectable moiety. A fluorescent in situ hybridization probe or a non-fluorescent in situ hybridization probe is typically from 15 bases to 2,000 bases in length (such as from 15-30 bp in length, from 15 bp to 100 bp in length, from 30 bp to 100 bp in length, from 100 bp to 1000 bp in length, from 500 bp to 200 bp in length, etc.) though can be shorter or longer. An in situ hybridization bait portion of a (of a photoreactive) probe can be useful for (can be configured to) hybridizing with prey or target. After in situ hybridization bait portion of a (photoreactive) probe binds to its prey, light activation can be used to photoactivate the photoreactive probe, and the primary subject probe can be acted upon by the photoactivated probe to form a photoexcited primary subject probe, and the photoexcited primary subject probe can (or is configured to form) a covalent bond with a second molecule (a third molecule, a fourth molecule, etc.) in the biological sample, such as a biomolecule, such as carbohydrates, lipids, nucleic acids, and proteins. In some embodiments, a bait molecule can include a reactive ligand. The reactive ligand can be configured to react with a corresponding molecule in a sample of interest. Such a reaction can result in rapid and irreversible binding between the reactive ligand and the corresponding molecule. In some embodiments, a reactive ligand bait molecule can utilize self-labeling protein (SLP) technology. Self-labeling protein technology is based on the formation of a specific, covalent bond between a pair of reactants: a reactive ligand and a fusion protein configured to (engineered to) bind to the ligand. A fusion protein for use in self-labeling protein technology can be a self-labeling protein tag (typically derived from an enzyme) and fused to a protein of interest (POI). In contrast to enzymatic reactions that increase a rate of chemical reaction without themselves being permanently altered by the reaction, self-labeling protein technology can result in formation of a specific, covalent bond between the ligand and a corresponding fusion protein. Similar as to other enzymatic reactions, the rate of a reaction utilizing self-labeling protein technology is fast and the reaction is very specific. A ligand can label a fusion protein in self-labeling protein technology without the need for additional enzymes for the labeling. Examples of reactive ligands useful as bait (in a photoreactive probe) include but are not limited to a CLIP-tag™ ligand (also referred to as a CLIP-tag™ substrate or a CLIP-Tag™), a HaloTag® ligand (also referred to as a HaloTag® substrate or a HaloTag®), and a SNAP-tag® ligand (also referred to as a SNAP-tag® substrate or SNAP-tag®). An example of a SNAP-tag® ligand useful as bait is a benzylguanine (BG) derivative and an example of a corresponding fusion protein is a derivative of the 20 kDa DNA repair protein 06-alkylguanine-DNA alkyltransferase (hATG) (e.g., a self-labeling protein SNAP-tag protein portion) that reacts specifically and rapidly with benzylguanine (BG) derivatives and fused to a protein of interest. A SNAP-tag® reaction can give off chemically inert guanine and this (and other self-labeling protein systems) can be safely used with living cells, in addition to being useful with other samples, such as non-living (fixed) cells, cell extracts, or other tissue extracts. An example of a CLIP-tag™ ligand useful as bait is a benzylcytosine (BC) derivative such as O2-benzylcytosine with a cytosine leaving group via a benzyl linker. An example of a corresponding fusion protein is a derivative of the 20 kDa DNA repair protein 06-alkylguanine-DNA alkyltransferase (hATG) (e.g., a self-labeling protein CLIP-tag protein portion) that reacts specifically and rapidly with benzylcytosine (BC) derivatives and fused to a protein of interest. An example of a HaloTag® ligand useful as bait is a reactive haloalkane (e.g., chloroalkane) derivative and an example of a corresponding fusion protein is a modified version of a bacterial dehalogenase enzyme (e.g., a self-labeling protein HaloTag protein portion) that removes halogens from aliphatic hydrocarbon molecules such as through a nucleophilic aspartate residue fused and fused to a protein of interest. As a part of some photoreactive probes, a reactive ligand bait molecule is bound through a photoreactive probe linker (or a single chemical bond) to a photoreactive moiety of the photoreactive probe (e.g., as part of the “G” in a G-C-B photoreactive probe, a G-K2-K1-B photoreactive probe, etc.). A protein of interest can be an endogenous protein or a non-endogenous protein (an antibody, a structural protein, etc.). A fusion protein having a protein of interest can be tagged with a self-labeling protein portion (CLIP-tag protein portion, HaloTag protein portion, SLIP-tagged protein portion) and expressed using standard recombinant protein expression techniques and used as described herein. Cells can be genetically modified with a nucleic acid comprising a nucleotide sequence encoding the fusion protein and analyzed directly or a fusion protein can be expressed and added to a sample, such as to a cell sample, a cell extract sample, a tissue extract sample, etc. In some variations, rather than a fusion protein, a self-labeling protein can include a post-translation modification tag portion or a non-covalently protein portion connected to a protein of interest.

Prey for an in situ hybridization bait portion can be target DNA, target cDNA, or target RNA, such as part of a genome (e.g., a chromosome, expressed RNA, rRNA, etc.) in a cell, cell extract, or other tissue extract.

The concentration of a photoreactive probe can range from 0.1 ug/mL to 100 ug/mL, while the concentration of the subject probe can range from 1 uM to 20 mM. A wavelength of light for activation of the photoreactive probe or photoselective tagging and labeling ranges in some embodiments from about 200 nm to about 1600 nm, e.g., from about 200 nm to about 250 nm, from about 250 nm to about 300 nm, from about 300 nm to about 350 nm, from about 350 nm to about 400 nm, from about 400 nm to about 450 nm, from about 450 nm to about 500 nm, from about 500 nm to about 550 nm, from about 550 nm to about 600 nm, from about 600 nm to about 650 nm, from about 650 nm to about 700 nm, from about 700 nm to about 750 nm, from about 750 nm to about 800 nm, from about 800 nm to about 850 nm, from about 850 nm to about 900 nm, from about 900 nm to about 950 nm, from about 950 nm to about 1000 nm, from about 1000 nm to about 1100 nm, from about 1100 nm to about 1200 nm, from about 1200 nm to about 1300 nm, from about 1300 nm to about 1400 nm, from about 1400 nm to about 1500 nm, or from about 1500 nm to about 1600 nm. In some embodiments, a wavelength of light for performing photoselective tagging and labeling ranges from about 700 nm to about 1600 nm (near infrared light) (e.g., 780 nm) at two-photon light source; or ranges from about 300 nm to about 650 nm or to about 700 nm (visible light) (e.g., 360 nm, 405 nm, 425 nm) at single-photon light source. Wavelengths of light used for photoactivation of a photoreactive probe are generally different from the wavelengths of light used for imaging. In some embodiments, activation of the photoreactive probe utilizes optical radiation (light) at from around 300-450 nm, 550 nm for single photon activation or >720 nm for multiphoton activation. The particular wavelength depends on the particular photoreactive moiety of the photoreactive probe.

### Methods

Also disclosed herein are methods of photoselectively tagging and labeling biomolecules and analytical methods. The methods may be used to tag and/or label carbohydrates, lipids, nucleic acids, proteins, either alone or in combination. The methods may include the step of treating a biological sample with (subjecting a biological sample to) a photoreactive probe having a bait molecule and a photoreactive moiety and binding the bait molecule to a first molecule (i.e., a prey) in the biological sample. In some embodiments, the biological sample comprises a plurality of cells. In some embodiments, the biological sample comprises a plurality of living cells. In some embodiments, the biological sample includes at least one, at least 100, at least 1000 or at least 10,000 live cells. In some embodiments, the biological sample comprises cell extracts. In some embodiments, the photoreactive moiety is coupled to the bait molecule through a chemical bond or a linker. In some embodiments, the photoreactive probe and the first molecule form a non-covalently conjugated probe-first molecule complex or molecule. In some embodiments, the bait molecule comprises an antibody, a CLIP-tag, HaloTag, a SNAP-tag, protein A, protein G, protein L, protein A/G, protein A/G/L, immunoglobulin binding peptides, biotin binding protein(s) (such as avidin, streptavidin, neutravidin), an RNA molecule, a small molecule, a nucleic acid molecule, a fluorescent in situ hybridization (FISH) probe, fragment antigen binding region, or nanobody. In some embodiments, a photoreactive moiety or a photoreactive probe includes ruthenium-based photocatalyst, iridium-based photocatalyst, Rose Bengal derivatives, fluorescein derivatives, Methylene blue derivatives, Flavin derivatives, Lumiflavin, riboflavin, Pterin derivatives, photosensitized protein, miniSOG, Killer Red, phenol, aryl azide, or benzophenone.

The methods may include the step of illuminating the biological sample with an imaging lighting source of an image-guided microscope system. The methods may include the step of imaging the illuminated sample with a camera. The methods may include the step of acquiring with the camera at least one image of subcellular morphology of the sample in a first field of view. The methods may include the step of processing the at least one image and determining a region of interest in the sample based on the processed image. The methods may include the step of obtaining coordinate information of the region of interest.

The methods may include the step of delivering a subject probe to the biological sample. The methods may include the step of selectively illuminating with optical light the region of interest based on the obtained coordinate information to thereby activate a photoreactive moiety, resulting in covalent bond formation between a subject probe and the neighboring molecules or molecules of interest in a biological sample in a region of interest in a biological sample. The methods may include the step of removing unconjugated photoreactive probe from the biological sample so as to allow the photoreactive probe to guide the selectively illumination on the selected region of interest. In some embodiments, the step of selectively illuminating comprises illuminating a region for 25 us/pixel to 400 us/pixel, for 50 us/pixel to 300 us/pixel, for 75 us/pixel to 200 us/pixel, or for 400 us/pixel to 5000 us/pixel. In some embodiments, a primary subject probe includes a tag portion and a subject moiety. In some embodiments, a tag portion of the primary subject probe includes a biotin derivative, a click chemistry tag, a HaloTag, a SNAP-tag, a CLIP-tag, digoxigenin, nucleic acid tag, or a peptide tag. In some embodiments, the click chemistry tag includes an alkyne-based or azide-based moiety. In some embodiments, a subject moiety of a subject probe is configured to generate a reactive intermediate (e.g., phenoxyl radical, carbene or the like) (in a biological specimen) that is responsible for covalent bond formation with amino acids of proteins in proximity to a molecule of interest. In some embodiments, an activated photoreactive moiety can promote formation of a free radical in a subject probe to thereby form covalent bond between the photoreactive moiety with an amino acid or a biomolecule of the biological sample in the selected region of interest. In some embodiments, the amino acid (e.g., of protein of interest or neighboring protein molecule) may be alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine or the like.

The methods may further include a step of conjugating a detectable label with a subject probe and detectably proximity labeling neighbors proximal the first molecule (i.e., the prey) with detectable label activity. In some embodiments, the detectable label includes a catalytic label. In some embodiments, the detectably proximity labeling comprises photoselective proximity labeling a region less than 5 μm, less than 2 μm, less than 1 μm, less than 500 nm, less than 300 nm, less than 200 nm, or less than 100 nm in diameter (or in a longest dimension). Some embodiments include a step of conjugating a connector with the subject probe. In some embodiments, the connector is conjugated to the subject probe through affinity between the connector and the primary subject probe so as to identify the location of the biological sample covalently bound with the subject probe. Some embodiments further include a step of conjugating a tag-peroxidase to the connector. In some embodiments, the tag-peroxidase is configured to catalyze an additional subject probe (e.g., a tyramide probe) to form a covalent bond between the additional subject probe and the biological sample. (The subject probe may be a first subject probe and the additional subject probe may be a second subject probe). More specifically, the tag-peroxidase can activate the additional subject probe to have a free radical and form the covalent bond between the additional subject probe and a tyrosine of the biological sample. Some embodiments further include the step of removing at least the region of interest from the microscope stage. Some embodiments further include the step of subjecting the selectively illuminated sample to mass spectrometry or sequencing analysis.

Some methods include contacting a biological sample having a target biomolecule with a photoreactive probe as described herein to non-covalently conjugate a photoreactive probe of the kit with the target biomolecule, washing unconjuagated photoreactive probe away, using optical radiation to spatially selectively activate the photoreactive probe of the kit and thus to induce the bonding between a tag-bearing subject probe of the kit and a nearby molecule in proximity to the target biomolecule, washing unbound subject probe away, and further comprises the step of labeling the tag-bearing biomolecule/probe complex with a label, and selectively proximity labeling neighbors proximal the target biomolecule.

Also disclosed herein are mass spectrometry-implemented methods for processing photo-labeled sample to predict a biomarker. The methods may include the step of dividing into a photolabeling sample group and a non-labeling sample group from a plurality of biological samples. The methods may include the step of delivering the photoreactive kit as described herein to the photolabeling sample group and the non-labeling sample group. In some embodiments, the bait molecule is non-covalently conjugated to a first molecule in the biological samples. The methods may include the step of selectively illuminating a selected region of interest of the photolabeling sample group and keeping the non-labeling sample group in the dark, wherein the illuminating step allows the primary subject probe to form a covalent bond with the biological sample. In some embodiments, the step of selectively illuminating the selected region of interest further comprises the step of activating the photoreactive moiety in the selected region so as to allow the activated photoreactive moiety to allow the primary subject probe to form the covalent bond with the biological sample in the selected region of interest. The methods may include the step of removing unconjugated photoreactive probe from the biological sample so as to allow the photoreactive probe to guide the selectively illumination on the selected region of interest. In some embodiments, the activated photoreactive moiety can activate the primary subject probe to have a free radical and form the covalent bond with an amino acid or a biomolecule of the biological sample in the selected region of interest. In some embodiments, the amino acid may be alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine.

The methods may include the step of extracting a plurality of the subject probe-bound proteins from the photolabeling sample group and the non-labeling sample group through an affinity precipitation between the primary subject probe-bond proteins and a plurality of affinity beads (e.g., streptavidin magnetic beads). The methods may include the step of subjecting the extracted proteins to mass spectrometry analysis. The methods may include the step of calculating a relative quantification value of an individual protein in an identified protein list between the photolabeling sample group and the non-labeling sample group according to an intensity value of a peptide fragment of the individual protein. The methods may include the step of determining a threshold of the relative quantification value between the photolabeling sample group and the non-labeling sample group. The methods may include the step of upon determining the threshold, predicting at least one biomarker corresponding to the relative quantification value of the individual protein over the threshold.

Also disclosed herein are mass spectrometry-implemented methods for processing photo-labeled sample to identify a biomarker list. The methods may include the step of delivering the photoreactive kit as described herein to a biological sample. In some embodiments, the bait molecule is non-covalently conjugated to a first molecule in the biological samples. The methods may include the step of selectively illuminating a selected region of interest of the biological sample so as to allow the primary subject probe to label a protein of the biological sample at the selected region of interest. In some embodiments, the step of selectively illuminating the selected region of interest further comprises a step of activating the photoreactive moiety in the selected region so as to allow the activated photoreactive moiety to allow the primary subject probe to form the covalent bond with the biological sample in the selected region of interest. The methods may include the step of removing unconjugated photoreactive probe from the biological sample so as to allow the photoreactive probe to guide the selectively illumination on the selected region of interest. In some embodiments, the activated photoreactive moiety can activate the primary subject probe to have a free radical and form the covalent bond with an amino acid or a biomolecule of the biological sample in the selected region of interest. In some embodiments, the amino acid may be alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine.

The methods may include the step of extracting a plurality of the probe-labeled proteins from the biological sample through an affinity precipitation between the primary subject probe-bond proteins and a plurality of affinity beads (e.g., streptavidin magnetic beads). The methods may include the step of subjecting the extracted proteins to mass spectrometry analysis. The methods may include the step of identifying the extracted proteins of the biological sample. The methods may include the step of calculating an intensity value of a peptide fragment of each protein from an identified protein list of the biological sample. The methods may include the step of ranking the identified protein list according to the intensity value of each protein.

The methods may include identifying and/or purifying one or more biomarkers (e.g., proximal or neighboring molecules).

For predicting a biomarker or identifying a biomarker list, the methods may further include the step of delivering a connector to the biological sample and conjugating the connector to the primary subject probe-bond proteins or biomolecules through the affinity between the connector and the primary subject probe. In some embodiments, the connector can be a fluorescent connector so as to identify the location of the biological sample covalently bound with the primary subject probe. The methods may further include the step of delivering a tag-peroxidase to the biological sample and conjugating the tag-peroxidase to the connector so as to allow the tag-peroxidase to catalyze an additional subject probe (e.g., tyramide probe) to form a covalent bond between the additional subject probe and the biological sample. In some embodiments, the tag-peroxidase activates the additional subject probe to have a free radical and form the covalent bond between the additional subject probe and a tyrosine of the biological sample.

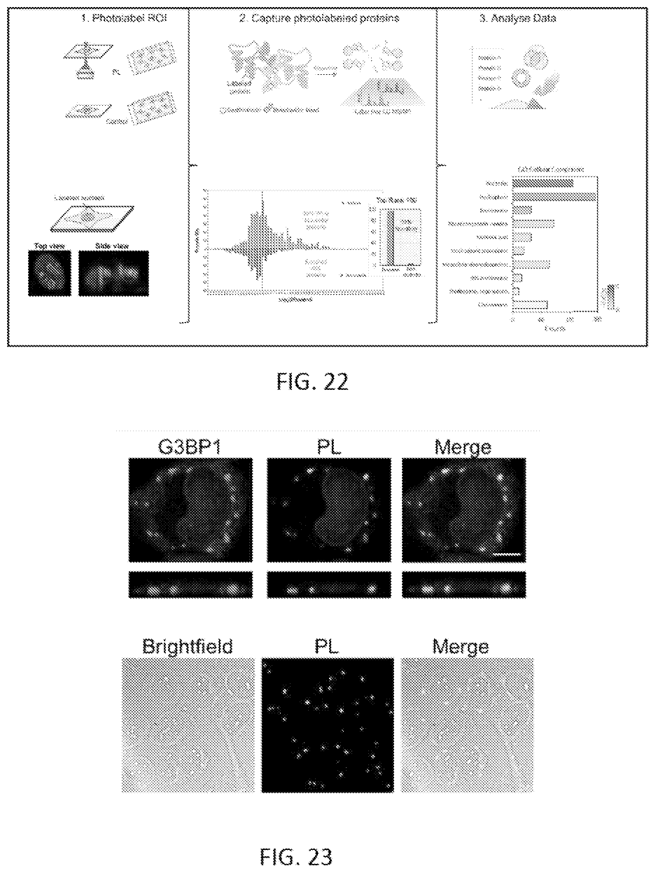

FIG. 22 illustrates a workflow for use of a photoreactive kit as described herein followed by mass spectrometry analysis. Workflow of the photoactive kit coupled with a microscopic photolabeling system followed by mass spectrometry analysis. Photolabeled nucleoli were harvested for LC-MS/MS analysis (bottom left). Protein distribution of true positive of nucleolar proteome. Proteins were ranked by order of ratios (two photon label/control), 97 out of the top 100 proteins were annotated as nucleolar proteins (bottom middle). Gene ontology analysis of nucleolar proteome (bottom right). Protocol: see [LC-MS/MS analysis] and [Protein identification and label-free quantification] below.

### EXPERIMENTAL AND METHODS

[Conjugation of ruthenium-based antibody] Ru(bpy)₃ NHS-ester (Bis(2,2′-bipyridine)-4′-methyl-4-carboxy bipyridine-ruthenium N-succinimidyl ester-bis(hexafluorophosphate) [CAS No.: 136724-73-7] were conjugated to the donkey anti-rabbit/donkey anti-mouse IgG antibodies via an amide coupling reaction, as shown in FIG. 7. Three hundred micrograms of antibodies were reacted with Ru(bpy)₃ NHS-ester in a final condition of 0.5 mg/mL antibody and 0.35 mM of Ru(bpy)₃ NHS-ester in 100 mM borate buffer (pH 8.0). The reactions were performed at room temperature for 2 h in the dark. Glycine was added to 100 mM for inactivating the reactions, and the antibody-Ru(bpy)₃ conjugates were then purified by an off-line size exclusion column (PD midiTrap G-25, Cytiva) with a gravity flow. The concentrations of purified antibody-Ru(bpy)₃ conjugates were measured by Pierce™ BCA protein assays (Thermo Fisher Scientific) using the unconjugated antibodies as the standards. For confirming that the Ru(bpy)₃ molecules is conjugated on the antibodies, the purified products were further detected by a NanoDrop spectrophotometer (Thermo Fisher Scientific) at 455 nm and 280 nm wavelength.

[Cell preparation] Cells were cultivated at 37° C. in a 5% CO2 humidified environment in Dulbecco's Modified Eagle Medium supplemented with 10% FBS. 2×105 cells were seeded in glass bottom chambers and incubated for approximately 16 h to 80-90% of confluency. Afterwards, cells were washed with PBS and fixed with 2.4% paraformaldehyde (PFA) or methanol. Fixed cells were incubated with PBS/0.5% Triton X-100 to permeabilize the cell membrane, and blocked with 3% BSA in PBS/0.1% Triton X-100 for 1 h, followed by 30 min of 0.002% streptavidin and 15 min of 40 μM biotin blocking.

[Hybridization of ruthenium-based antibody] Cells were incubated with primary antibody for 2 h at room temperature with the following antibodies: rabbit anti-NCL, mouse anti-G3BP1, mouse anti-NPC in blocking buffer [PBS/0.1% Triton X-100 with 3% BSA]. After washing with PBS/0.1% Triton X-100, 10 μg/mL of antibody-Ru(bpy)₃ conjugates were hybridized to the primary antibody for overnight at 4° C. Cells were subsequently stained with fluorescence marker (goat anti-rabbit AlexaFluor 647 or goat anti-mouse AlexaFluor 647) for 1 h at room temperature.

[Two-photon labeling on subcellular cell for quantitative LC-MS/MS analysis] Cells were incubated with two-photon (2P) labeling reagent containing 5-7 mM desthiobiotin-phenol and 0.005% methyl viologen. Two-photon laser coupled with a microscopic system was used for spatially resolved photolabeling at a laser power of 100-200 mW, and the cells were subjected to a laser-exposure time at 100-1000 microseconds. Labeled cells were washed with the buffer containing 10 mM sodium ascorbate, 5 mM trolox, and 0.02% sodium azide for quenching the photochemical reaction, and cells were washed with PBST three times in final.

[Verification of the performance of two-photo labeling by fluorescent microscopy] Labeled cells/signals were probed with neutrAvidin-DyLight 488 for 1 h with blocking buffer containing 3% BSA/PBS/0.1% Triton X-100, and the antibody-Ru(bpy)₃ conjugates were hybridized with fluorescence marker (goat anti-donkey TexasRed) or anti-primary antibody (goat anti-rabbit-568 or goat anti-mouse-568) for 1 h at room temperature. Cells were subsequently stained with nuclear marker (Hoechst 33258) for 30 min at room temperature. A Zeiss LSM 880 confocal microscope was applied to verify that the labeling signals are within the boundaries of the organelles at xy-sections and xz-sections.

FIG. 8 shows experimental results of antibody-based ruthenium (Ab-Ru) targeted photo-labeling (confocal imaging of nucleolar markers (magenta), antibody-based ruthenium (red), 2P labeled signals: desthiobiotin (green); scale bar: 20 μm). FIG. 8 shows experimental results of antibody-based ruthenium (Ab-Ru) targeted photo-labeling (confocal imaging of nucleolar markers (magenta), antibody-based ruthenium (red), 2P labeled signals: desthiobiotin (green); scale bar: 20 μm).

[Protein extraction and on-bead digestion] Labeled cells were harvested by scraping with buffer containing 10 mM Tris (pH 8.0), 1% Triton X-100, 1-fold protease inhibitor cocktail, 10 mM sodium ascorbate, 5 mM Trolox, and 1 mM sodium azide. Harvested cells were sonicated at 60% power using a Q125 sonicator (Qsonica) with 1 s on/2 s off interval for 2 min, then subjected to evaporate the scraping buffer for 2 h by SpeedVac system. 160 μL of lysis buffer containing 4% Sodium dodecyl sulfate (SDS), 1% Triton X-100, 100 mM Tris (pH 8.0), and 20 mM dithiothreitol (DTT) were added to the harvested cells, and the mixture was vortexed at 1 min on/2 min off interval for 5 cycles. To retrieve the cross-linked amide groups resulting from PFA fixation, the lysed cells were further heated at 99° C. for 45 min followed by another round of vortexing at 1 min on/2 min off interval for 5 cycles. Lysates were centrifuged at 16,000 g for 20 min at 20° C., and the supernatant was collected. Pierce™ 660 nm Protein Assay (Thermo Fisher Scientific) was used to measure the protein concentrations, and 240 μg of protein were subjected to immunoprecipitation. Streptavidin magnetic beads were washed with dilution buffer [0.5% Triton X-100/PBS] three times, and the protein lysates were diluted 10-fold to reduce the SDS concentration to be less than 0.4%, and the diluted lysates were added to the washed beads and incubated at 2-8° C. for 16 h with rotation. After which, the biotin-protein bonded beads were washed with the following washing buffers to reduce the non-specific binding maximally: Buffer A [2% SDS, 50 mM Tris (pH 8.0)]; Buffer B [0.5M NaCl, 0.1% deoxycholic acid, 0.1% SDS, 1% Triton X-100, 50 mM HEPES]; Buffer C [0.5% deoxycholic acid, 0.5% Triton X-100, 10 mM Tris (pH 8.0), 250 mM LiCl]. For on-bead digestion, the beads were further washed with 100-μL 50 mM triethylammonium bicarbonate buffer three times, and the biotin-protein bonded beads were then mixed with 0.2 μg of Trypsin/Lys-C(V5071, Promega) in a final volume of 20 μL at 37° C. for 100 min for an initial digestion. After that, the supernatants were collected and subjected to the overnight digestion without adding further enzyme. Finally, the digests were acidified by adding 2 μL of 10% formic acid, and were desalted by C18 Ziptip. Desalted peptides were dried by Speedvac and stored at −20° C. prior to LC-MS/MS analysis.

[LC-MS/MS analysis] Detection of immunoprecipitated product by data-dependent acquisition mass spectrometry. LC-MS/MS analysis was performed using an UltiMate 3000 RSLCnano system (Thermo Fisher Scientific) coupled to an Orbitrap Fusion Lumos mass spectrometer (Thermo Fisher Scientific). The desalted peptides were resuspended in 0.1% formic acid in water and loaded onto a PepMap™ 100 C18 HPLC column (2 μm, 100 angstrom, 75 μm×25 cm; Thermo Fisher Scientific), and peptides were eluted over 160 min gradients for nuclei-illuminated samples, over 120 min gradients for nucleoli-, SG-illuminated samples. The full MS spectra ranging from m/z 375-1500 were acquired at a resolving power of 120,000 in Orbitrap, an AGC target value of 4×105, and a maximum injection time of 50 ms. Fragment ion spectra were recorded in the top-speed mode at a resolving power of 30,000 in Orbitrap using a data-dependent method. Monoisotopic precursor ions were selected by the quadrupole using an isolation window of 1.2, 0.7, 0.4 Th for the ion with 2+, 3+, 4-7 charge states, respectively. An AGC target of 5×104, maximum injection time of 54 ms, higher-energy collisional dissociation (HCD) fragmentation with 30% collision energy, and a maximum cycle time of 3 s were all applied. Dynamic exclusion was set to 60 s with an exclusion window of 10 ppm. Precursor ions with the charge state of unassigned, 1+, or superior to 8+ were excluded from fragmentation selection.

[Protein identification and label-free quantification] Raw data from the same batch of two-photon illumination were processed together with Proteome Discoverer (Thermo Fisher Scientific) by Sequest HT algorithm against the UniProtKB/Swiss-Prot human protein database (version 2020.02, 20,365 entries) for feature extraction, peptide identification, and protein inference. Database search was performed as follows: tryptic peptides with up to three missed cleavages; mass tolerances of 10 ppm for peptide ions, and 0.05 Da for fragment ions; static carbamidomethylation (+57.0215 Da) on Cys residues; dynamic deamidation (+0.9840 Da) on Asp and Gln residues, oxidation (15.9949 Da) on Met residues, acetylation on protein N-termini (+42.0106 Da), and desthiobiotin phenol modification (+331.1896) on Tyr residues. The minimal peptide length was set as 6 residues. The false discovery rate (FDR) of peptide and protein were both set as 1%.

For label-free quantification, the time windows for chromatographic peak alignment was set as 20 min. Peptide level data was then normalized to the total peptide intensity, and the quantification value for a given protein was derived from the sum of normalized intensities of the top three intense unique peptides belonging to that protein.

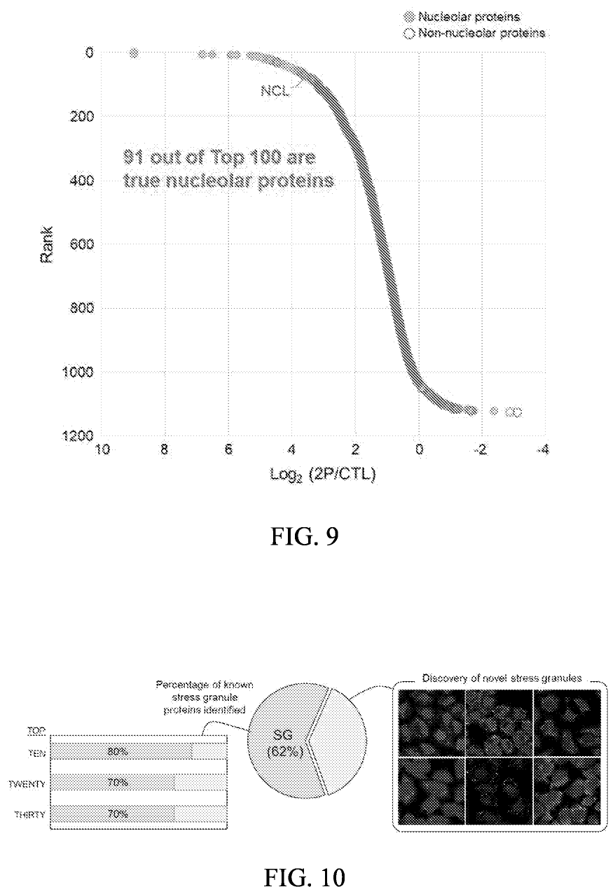

FIG. 9 shows results of nucleolar labeling experiment. FIG. 9 shows total identified proteins from nucleolar labeling experiment. 91% of top 100 proteins are nucleolar proteins, suggesting that the labeling of small ROIs (nucleoli) by Ab-Ru and subject probe (biotin-phenol) is accurate, and extraction steps and identification of tag (biotin)-labeled proteins result in excellent labeling.

Proteins are ranked by their fold-change ratio, labeled cells over unlabeled cells in logarithm. FIG. 10 shows that proteome composition reveals the accuracy and the capability to discover novel proteins for stress granules (SG). 62% of the enriched proteins are SG proteins, among the enriched proteins not included in the current SG database, at least six proteins were validated by immunocytochemistry to be co-localized with the SG, suggesting that the antibody-based ruthenium targeted photo-labeling can discover novel proteins of the labeled area.

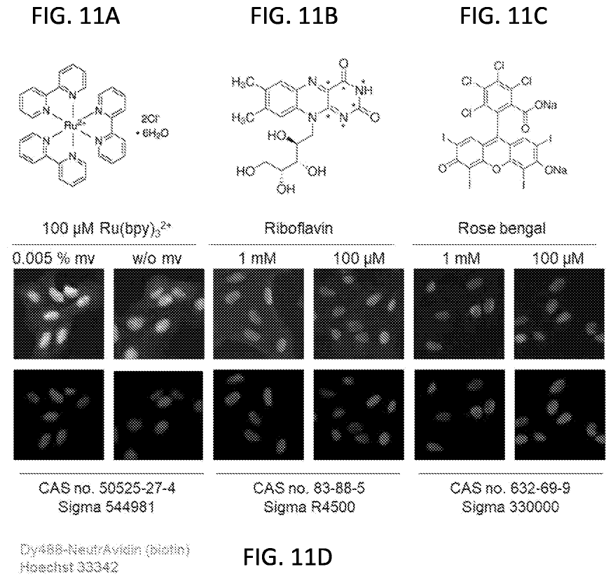

FIGS. 11A-C show structural formulations of three commercially available photoactivate probes: FIG. 11A: Ru(bpy)₃²⁺, FIG. 11B: riboflavin, and FIG. 11C: Rose Bengal (Top panel). Photolabeling was performed on an entire FOV (wavelength (λ)=780 nm, power=100 mW) that directly scanned onto U-2OS cells along with biotinyl tyramide. FIG. 11D shows that the highest fluorescent signals were detected using Ru(bpy)3²⁺ with 0.005% methyl viologen. Experimental protocol for FIG. 11: U-2OS cells were incubated with photolabeling reagent containing 500 μM desthiobiotin-phenol and free photoreactive moieties: Ru(bpy)32+, riboflavin, and rose bengal at a range from 100-1000 μM for 10 min at room temperature. Two-photon microscopy system was used for spatially resolved photochemical labeling on an entire field of view (FOV). Two-photon microscopic system was used for spatially resolved photolabeling at a laser power of 10-200 mW, and the cells were subjected to a laser-exposure time at 100-200 microseconds. Labeled cells were washed with the buffer containing 10 mM sodium ascorbate, 5 mM trolox, and 0.02% sodium azide for quenching the photochemical reaction, and cells were washed with PBST three times in final. Labeled cells were washed with the buffer containing 10 mM sodium ascorbate, 5 mM trolox, and 0.02% sodium azide for quenching the photochemical reaction, and cells were washed with PBST three times in final. Labeled cells/signals were probed with neutrAvidin-DyLight 488 for 1 h with blocking buffer containing 3% BSA/PBS/0.1% Triton X-100. Cells were subsequently stained with nuclear marker (Hoechst 33258) for 30 min at room temperature.

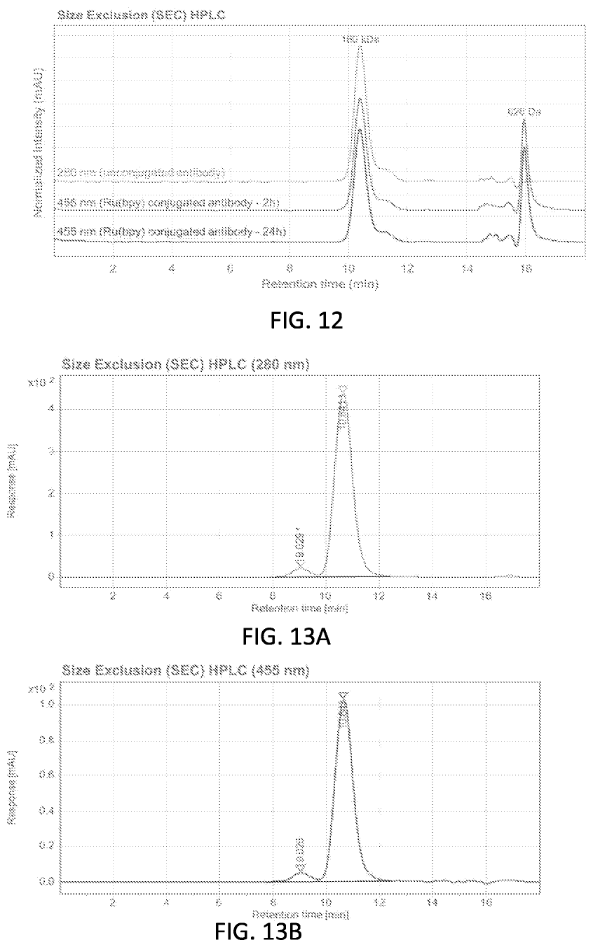

FIG. 12 shows successful conjugation of photoreactive moieties with antibody. 1 mg of antibodies were reacted with Ru(bpy)-NHS at 100-fold molar ratio for 2 h or 24 h, and the resultants were detected by SEC-HPLC to verify the conjugation of Ru(bpy)₃²⁺ molecules to antibodies. The SEC-HPLC showed Ru(bpy)₃²⁺ conjugated antibodies (160 kDa) have both characteristic peaks of 280 nm and 455 nm at 10.4 min, indicating a successful conjugation of Ru(bpy)₃²⁺ and antibody.

FIGS. 13A-13B shows results of successful conjugation of photoreactive moieties with antibody. A 10 mg scale of Ru(bpy)₃²⁺ antibody conjugation followed by removing unconjugated Ru(bpy)₃²⁺ molecules using 40 kDa zeba spin desalting columns. A major Ru(bpy)₃²⁺ conjugated antibody peak was observed at both 280 nm and 455 nm absorbance. No free Ru(bpy)₃²⁺ was found in 16 min at 455 nm absorbance. Protocol for FIGS. 12 and 13A-13B: see 1) “[Conjugation of ruthenium-based antibody]” (above) and 2): “[SEC-HPLC protocol (to verify Ab-Ru conjugates]” The purities of the unconjugated antibodies and Ru(bpy)-conjugated antibodies (Ab-Ru) under native state were determined by SEC-HPLC. The mobile phase was sodium phosphate buffer, pH 7.4. Test samples were diluted with mobile phase to 1 mg/mL, and 100 μL of the resultants were injected into a HPLC system equipped with TSKgel G3000SWXL column, 7.8 mm ID×30 cm. An isocratic flow of mobile phase at 1.0 mL/min was applied to the protein separation. The characteristics of the eluents to be monitored were unconjugated antibodies (160 kDa), Ab-Ru (160 kDa), aggregates (>160 kDa), and free Ru(bpy) molecules (628 Da). The unconjugated antibodies and aggregates were detected by 280 nm, Ab-Ru and free Ru(bpy) molecules were detected by 455 nm. All the measurements were conducted by a photodiode array detector.

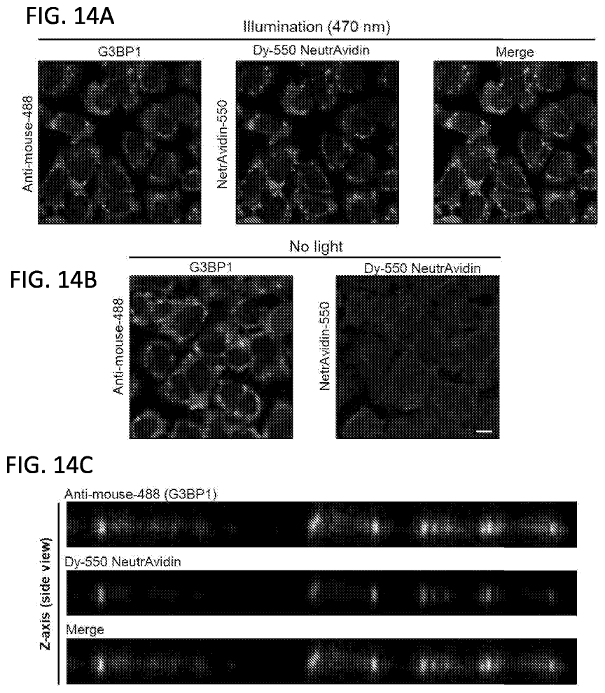

FIGS. 14A-14C show results from specific photolabeling of induced cells using conjugation of a ruthenium based photoreactive moieties with antibody. FIG. 14 shows experimental results of antibody-based ruthenium (Ab-Ru) targeted photo-labeling (confocal imaging of stress granule markers (green), photolabeled signals: desthiobiotin (red); scale bar: 10 μm). FIG. 14A shows illumination at 470 nm. FIG. 14B shows a control (no light). FIG. 14C shows a Z-axis (side view) of the cells shown in FIG. 14A. Specificity and high-resolution of the illumination effects on antibody conjugated Ru(bpy)₃²⁺ (Ab-Ru). U-2OS cells were induced by arsenite and hybridized with rabbit anti G3BP1 (stress granule marker: green) to show the region of interest for labeling. Donkey anti-mouse secondary antibody were conjugated with Ru(bpy)₃²⁺ (Ab-Ru photoactive probe) and illuminated at a wavelength of 470 nm LED along with desthiobiotin-phenol and methyl viologen. Clear stress granule patterns were observed after Dy-550 neutravidin staining. No light control groups showed no stress granule patterns after Dy-550 neutravidin staining. Z-axis images also showed the specificity and a high resolution of photolabeling effects in the z-plane using Ab-Ru photoactive probe. Protocol for FIGS. 14, 17, 18, 20, 22, 23, and 24: “Hybridization of ruthenium-based antibody” (see above), followed by “Label and analysis”: Cells were incubated with photolabeling reagent containing 5 mM desthiobiotin-phenol and 0.005% methyl viologen. 470 nm LED laser coupled with a microscopic system was used for spatially resolved photolabeling at a laser power of 10-200 mW, and the cells were subjected to a laser-exposure time at 37-1000 microseconds. Labeled cells were washed with the buffer containing 10 mM sodium ascorbate, 5 mM trolox, and 0.02% sodium azide for quenching the photochemical reaction, and cells were washed with PBST three times in final.

Labeled cells/signals were probed with neutrAvidin-DyLight550 for 1 h with blocking buffer containing 3% BSA/PBS/0.1% Triton X-100, and the mouse anti-G3BP1 antibody were hybridized with fluorescence marker (goat anti-mouse 488) for 1 h at room temperature. A Zeiss LSM 880 confocal microscope was applied to verify that the labeling signals are within the boundaries of the organelles at xy-sections and xz-sections.

FIG. 14 shows experimental results of antibody-based ruthenium (Ab-Ru) targeted photo-labeling (confocal imaging of stress granule markers (green), photolabeled signals: desthiobiotin (red); scale bar: 10 μm).

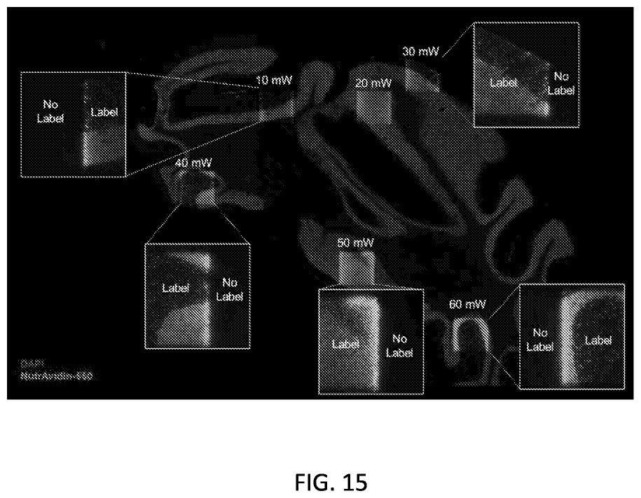

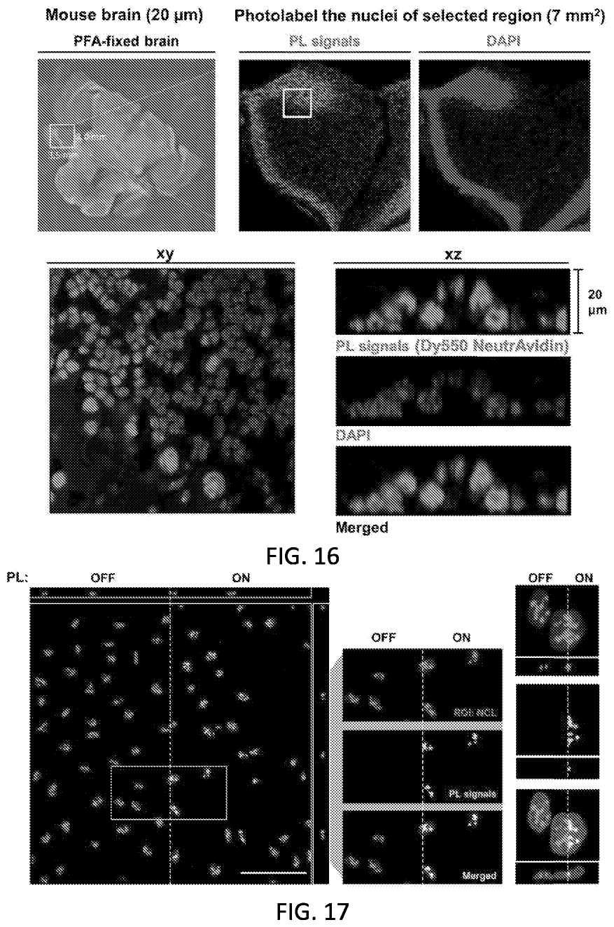

FIG. 15 shows selective photolabeling of mouse brain tissue samples using a probe of ruthenium based photoreactive moieties with antibody. Control (no light) shows no labeling. A 20 μM frozen mouse brain tissue section was stained with rabbit anti-nucleolin (nuclear marker) and then hybridized with anti-rabbit Ab-Ru photoactive probe (donkey anti-rabbit secondary antibody was conjugated with Ru(bpy)₃²⁺) and illuminated at a wavelength of 457 nm LED along with desthiobiotin-phenol and methyl viologen at power 10, 20, 30, 40, 50, 60 mW. Clear nuclear patterns were observed after Dy-550 neutravidin staining to show the photolabeling signals (desthiobiotin) on tissue at selected areas. No light control areas showed no labeling signals. Protocol for tissue preparation, primary antibody, and Ab-Ru staining for FIG. 15, 16, 19: “Tissue preparation, primary antibody, and Ab-Ru staining” PFA fixed mouse brain tissue was embedded in optimal cutting temperature (OCT) compound and cryosection onto a cover slide. The 10-30 μm mouse brain section was incubated with rabbit anti-NCL antibody in blocking buffer [PBS/0.1% Triton X-100 with 3% BSA] for overnight at 4° C. After washing with PBS/0.1% Triton X-100, 10 μg/mL of antibody-Ru(bpy)₃ conjugates were hybridized to the primary antibody for overnight at 4° C. Tissue section was subsequently stained with fluorescence marker goat anti-rabbit AlexaFluor 647 for 1 h at room temperature. Additional protocol for FIGS. 15 and 16: “457 LED light source”: 10-30 μm of tissue section was incubated with labeling reagent containing 5 mM desthiobiotin-phenol and 0.005% methyl viologen. 457 nm LED laser coupled with a microscopic system was used for spatially resolved photolabeling at a laser power of 10-200 mW, and the tissue section was subjected to a laser-exposure time at 37-1000 microseconds. Labeled tissue section was washed with the buffer containing 10 mM sodium ascorbate, 5 mM trolox, and 0.02% sodium azide for quenching the photochemical reaction, and then washed with PBST three times in final. Additional protocol for FIGS. 15, 16, 19: “Verification of photolabeling performance”: Labeled tissues/signals were probed with NeutrAvidin-DyLight 550 for 1 h with blocking buffer containing 3% BSA/PBS/0.1% Triton X-100. Tissue section was subsequently stained with nuclear marker (DAPI) for 30 min at room temperature. A fluorescent microscope or Zeiss LSM 880 confocal microscope was applied to verify that the labeling signals are within the boundaries of the nuclei at xy-sections and xz-sections.

FIG. 16 shows experimental results showing photolabeling of mouse brain tissue samples using a probe of ruthenium based photoreactive moieties with antibody and neutravidin staining. A 20 μM frozen mouse brain tissue section was stained with rabbit anti-nucleolin (nuclear marker) and then hybridized with anti-rabbit Ab-Ru photoactive probe (donkey anti-rabbit secondary antibody was conjugated with Ru(bpy)3²⁺) and illuminated at a wavelength of 457 nm LED along with desthiobiotin-phenol and methyl viologen. Clear nuclear patterns were observed after Dy-550 neutravidin staining. Z-axis images showed the specificity and a high resolution of photolabeling effects in the z-plane using antibody-Ru(bpy)3²⁺ photoactive probe.

FIG. 17 shows experimental results showing detection of nucleoli regions using a probe of ruthenium based photoreactive moieties with and a secondary antibody bait. Photolabeling signals were labeled using two-photon technique. Photolabeled (ON) and non-labeled (OFF) regions of interest were located within nucleoli (rabbit anti-NCL antibody: red) using Ab-Ru photoactive probe (donkey anti-rabbit secondary antibody was conjugated with Ru(bpy)3²⁺). Photolabeled (PL) signals were stained with anti-desthiobiotin (Dy-488 NeutrAvidin). PL signals were labeled using two-photon at wavelength=780 nm. Protocol for FIGS. 17, 18, 20, 22, 23, 24: 1). [Two-photon labeling on subcellular cell for quantitative LC-MS/MS analysis] (see above) and 2) [Verification of the performance of two-photo labeling by fluorescent microscopy] (see above).

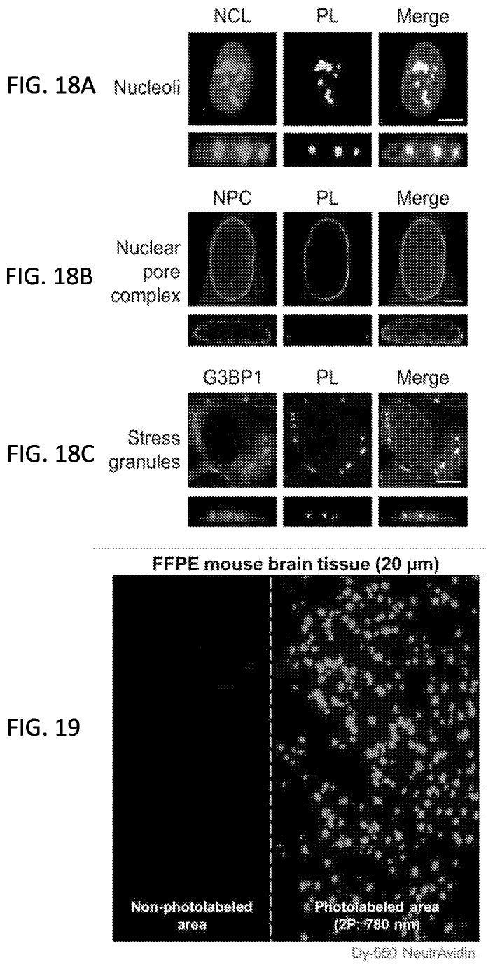

FIGS. 18A-18C show experimental results showing detection of photolabeled subcellular compartments using probe with ruthenium based photoreactive moieties and an Alexa fluor 568 secondary antibody bait to bind primary antibodies. FIG. 18A shows nucleoli detection using a rabbit anti-nucleolin antibody. FIG. 18B shows experimental results showing nuclear pore complex detection using a mouse anti-NPC antibody. FIG. 18C shows stress granule detection using a mouse anti-Ras GTPase-activating protein binding protein 1 antibody.

FIG. 19 shows experimental results showing specific 2 photon labeling on fixed mouse brain tissue. Protocol for FIG. 19 “2p Labeling”: 10-30 μm of mouse brain tissue section was incubated with two-photon (2P) labeling reagent containing 5-7 mM desthiobiotin-phenol and 0.005% methyl viologen. Two-photon laser coupled with a microscopic system was used for spatially resolved photolabeling at a laser power of 10-200 mW, and the tissue section was subjected to a laser-exposure time at 10-1000 microseconds. Labeled tissue section was washed with the buffer containing 10 mM sodium ascorbate, 5 mM trolox, and 0.02% sodium azide for quenching the photochemical reaction, and tissue section was washed with PBST three times in final.

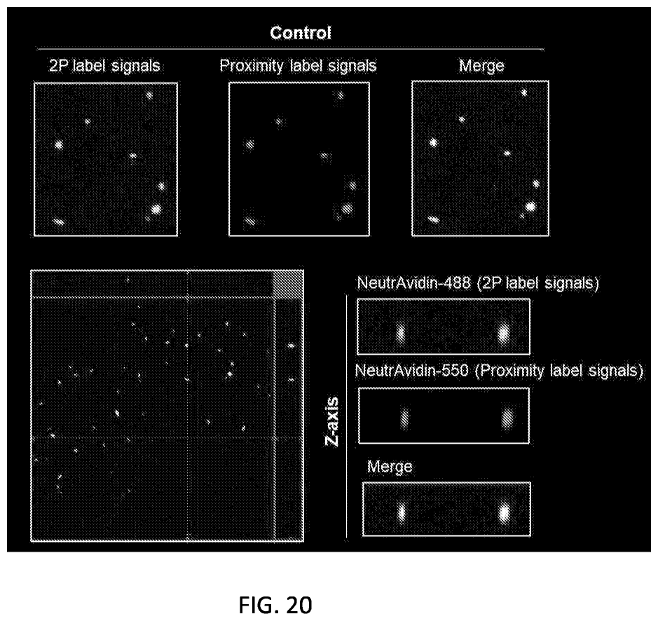

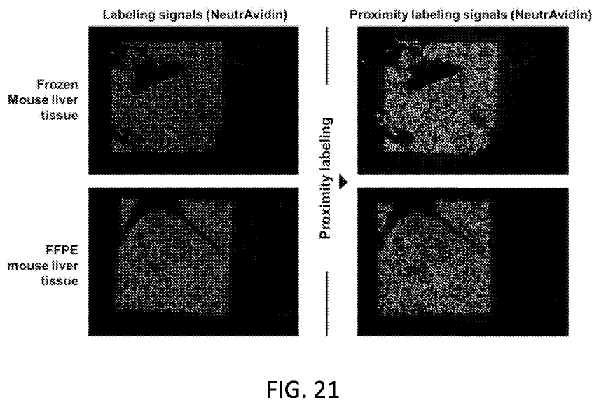

FIG. 20 shows experimental results showing photolabeled stress granules using horseradish peroxidase activated desthiobiotin to covalently bind to tyrosine residues on and proximal to the enzyme site. Protocol for FIGS. 20 and 21: “Proximity labeling after photolabeling”: Labeled cells/tissues/signals were probed with NeutrAvidin-DyLight488 or NeutrAvidin-DyLight550 for 1-2 h with blocking buffer containing 3% BSA/PBS/0.1% Triton X-100 at room temperature followed by anti-NeutrAvidin-HRP (biotin-HRP) hybridization for 1 h at room temperature. 100 μm of desthiobiotin was added and incubated in the cell/tissue samples for 30 min at room temperature. Hydrogen peroxide was added to catalyse HRP activated desthiobiotin to covalently bind to tyrosine residues on and proximal to the enzyme site. Proximity labeled signals were probed with NeutrAvidin-DyLight550 for 1 h with blocking buffer containing 3% BSA/PBS/0.1% Triton X-100 for 1-2 h at room temperature. A fluorescence microscope or Zeiss LSM 880 confocal microscope was applied to verify that the labeling signals are within the boundaries of the organelles at xy-sections and xz-sections.

FIG. 21 shows proximity labeling of photolabeled mouse liver tissue samples using horseradish peroxidase activated desthiobiotin to covalently bind to tyrosine residues on and proximal to the enzyme site.

FIG. 22 shows an example of a workflow using a photoactive kit coupled with a microscopic photolabeling system followed by mass spectrometry analysis as described herein.

FIG. 23 shows experimental results showing confocal micrographs depicting precise and accurate photolabeled (PL) stress granules by antibody-ruthenium photoactive probe using a two-photon labeling system at wavelength=780 nm.

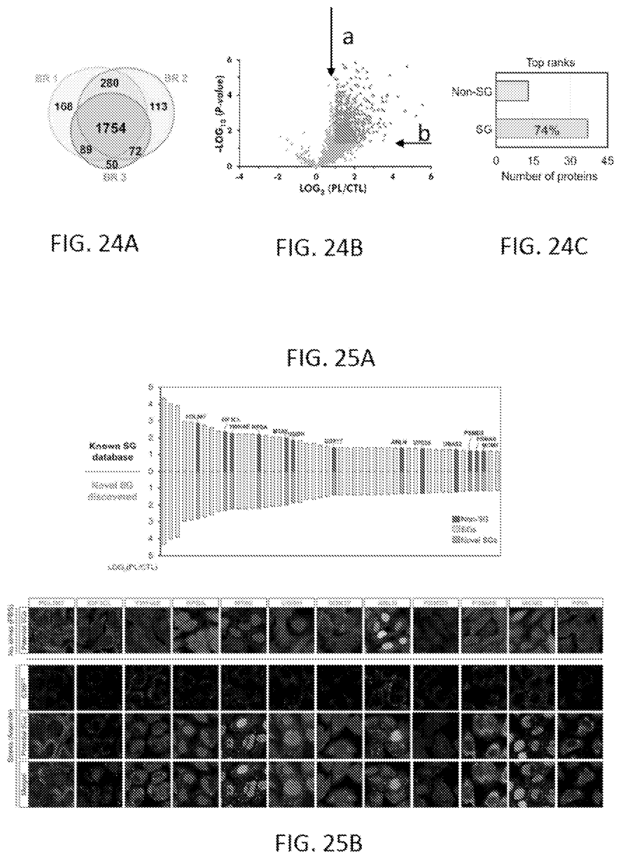

FIG. 24A-24C shows of two photon labeling using an antibody-ruthenium photoactive probe. FIG. 24A shows a Venn diagram of three biological replicates of two photon labeling Ab-Ru photoactive probe. FIG. 24B shows Volcano plot of relative protein levels in photolabeled samples to control samples (PL/CTL ratio) in a LOG2 scale. Over-represented (enriched) proteins are shown in the upper right bounded to arrow a and arrow b. FIG. 24C shows that 74% of true positive rate (annotated as stress granules (sg) are found in the top 50 proteins ranked by PL/CTL ratio. Protocol: see [LC-MS/MS analysis] and [Protein identification and label-free quantification] above.

FIG. 25A-25B show that 37 proteins in the top 50 proteins ranked by PL/CTL ratio were annotated as stress granule proteins (up). Validation of potential stress granules proteins by immune-fluorescent detection. Confocal micrographs depicting stress granule formation of potential stress granule proteins in U-2OS cells with or without arsenite stress. The potential stress granule proteins (green) are highly co-localized with well-known G3BP1 SG markers (red). The proteome composition reveals the accuracy and the capability to discover novel stress granule biomarkers using this method (bottom). Lens: 63× oil.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

1.-93. (canceled)

94. A photoreactive kit comprising:
a photoreactive probe represented by formula (I): 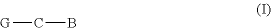
wherein
the C portion is a single chemical bond or a linker;
the B portion includes at least one photoreactive moiety bound to the C portion; and
the G portion comprises a bait molecule bound to the C portion; and
a primary subject probe comprising a detectable tag portion bound to a photoexcitable subject moiety,
wherein the bait molecule of the photoreactive probe is configured to conjugate with a target molecule in a sample, and
wherein, when the photoreactive probe is photoactivated and the primary subject probe is acted upon by the photoactivated probe to form a photoexcited primary subject probe, the photoexcited primary subject probe is configured to form a covalent bond with a nearby molecule in proximity to the target molecule in the sample.

95. The photoreactive kit of claim 94, wherein the bait molecule comprises at least one of an antibody, avidin, neutravidin, streptavidin, another biotin-binding protein, a CLIP-tag, a HaloTag, a SNAP-tag, another self-labeling protein tag, a DNA or RNA fluorescent in situ hybridization (FISH) probe, another RNA molecule, another nucleic acid molecule, protein A, protein G, protein L, protein A/G, protein A/G/L, another immunoglobulin binding peptide, a drug, or another small molecule.

96. The photoreactive kit of claim 94, wherein the bait molecule is an antibody and a number of the photoreactive moieties are bound to the antibody through the C portion, wherein the number ranges from 1 to 50.

97. The photoreactive kit of claim 94, wherein the photoreactive moiety is at least one of riboflavin, lumiflavin, another flavin derivatives, fluorescein or a derivative thereof, methylene blue or a derivative thereof, miniSOG photosensitized protein, Killer Red photosensitized protein, another photosensitized protein, pterin or a derivative thereof, a ruthenium-based photocatalyst, and Rose Bengal or a derivative thereof.

98. The photoreactive kit of claim 97, wherein the photoreactive moiety is derived from a ruthenium-based compound represented by formula (II): 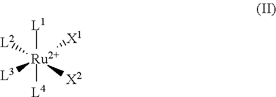
wherein in formula (II):
L¹, L², L³, and L⁴ are each independently a ligand; and
X¹ and X² are each independently a ligand, wherein at least one of X¹ and X² has linking region, wherein the at least one linking region is bound to the C portion of the photoreactive probe.

99. The photoreactive kit of claim 98, wherein the ruthenium-based compound of formula (II) is any of 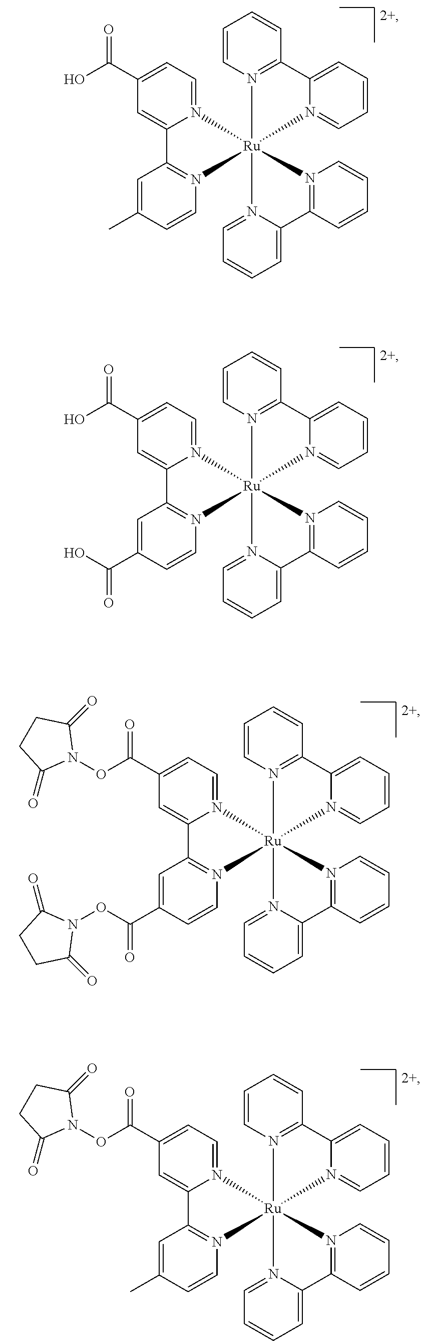 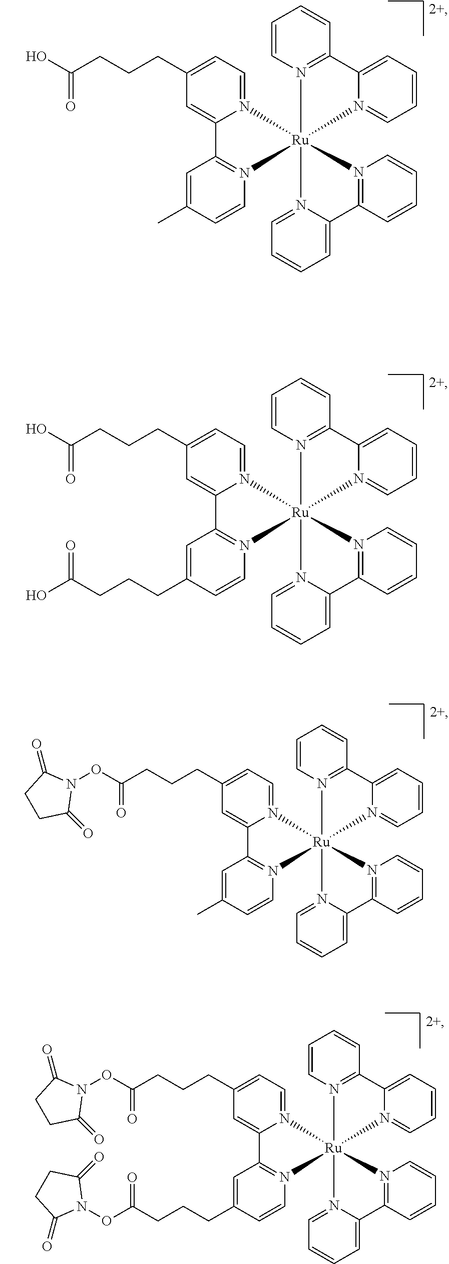 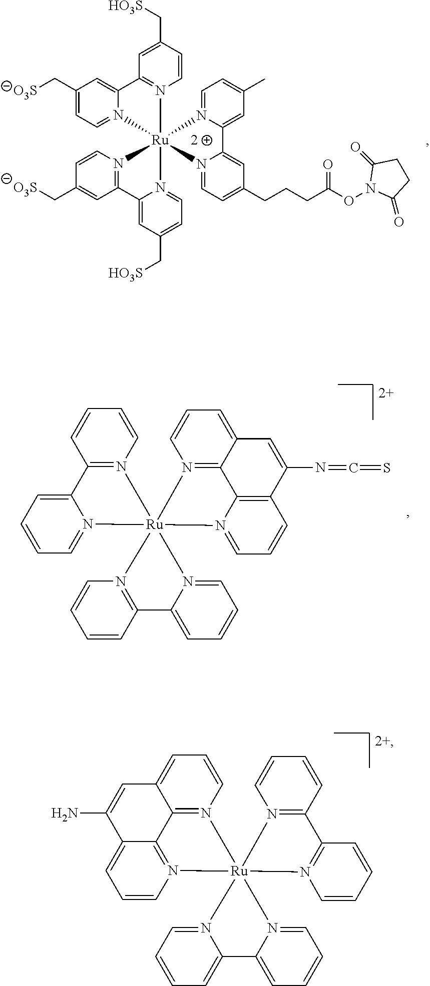
and derivatives thereof.

100. The photoreactive kit of claim 98, wherein X¹ and X² are each independently selected from the group consisting of 3-ethynylpyridine, 3-(bromomethyl)pyridine, maleimide, 4′-methyl-4-carboxybipyridine-N-succinimidyl ester, nicotinaldehyde, 1-(4-(pyridin-3-yl)-1H-1,2,3-triazol-1-yl) ethanone, 4-pentynenitrile, and 4-aminobutyne.

101. The photoreactive kit of claim 98, wherein L¹ and L² are joined to form a first bidentate ligand and L³ and L⁴ are joined to form a second bidentate ligand, wherein the first bidentate ligand and the second bidentate ligand are independently selected from the group consisting of 2,2′-bipyridyl (bpy), 4,4′-dicyano-5,5′-dimethyl-2,2′-bipyridine (CN-Me-bpy), 4,4′-dimethyl-2,2′-bipyridine (dmb), 4,4′-di-tert-butyl-2,2′-bipyridine (dbpy), 4,4′,5,5′-tetramethyl-2,2′-bipyridine (tmb), 2-phenylpyridine (ppy), 6-bromo-2,2′-bipyridine, 6,6′-dibromo-2,2′-bipyridine, 5-bromo-2,2′-bipyridine, 6-amino-2,2′-bipyridine, 6,6′-diamino-2,2′-bipyridine, 2,2′-bipyridine-6-carbonitrile, 2,2′-bipyridine-6,6′-bis(carbonitrile), 2,2′-bipyridine-6-carboxylic acid, 2,2′-bipyridine-6,6′-dicarboxylic acid, and biquinoline.

102. The photoreactive kit of claim 94, wherein the photoexcitable subject moiety is

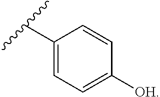 [SMILES: *c1ccc(O)cc1]

103. The photoreactive kit of claim 94, wherein the detectable tag portion is at least one of a biotin derivative, a click chemistry tag, a CLIP-tag, a digoxigenin tag, a HaloTag, an oligonucleotide, a peptide tag, and a SNAP-tag.

104. The photoreactive kit of claim 94, wherein the photoreactive moiety includes the moiety of 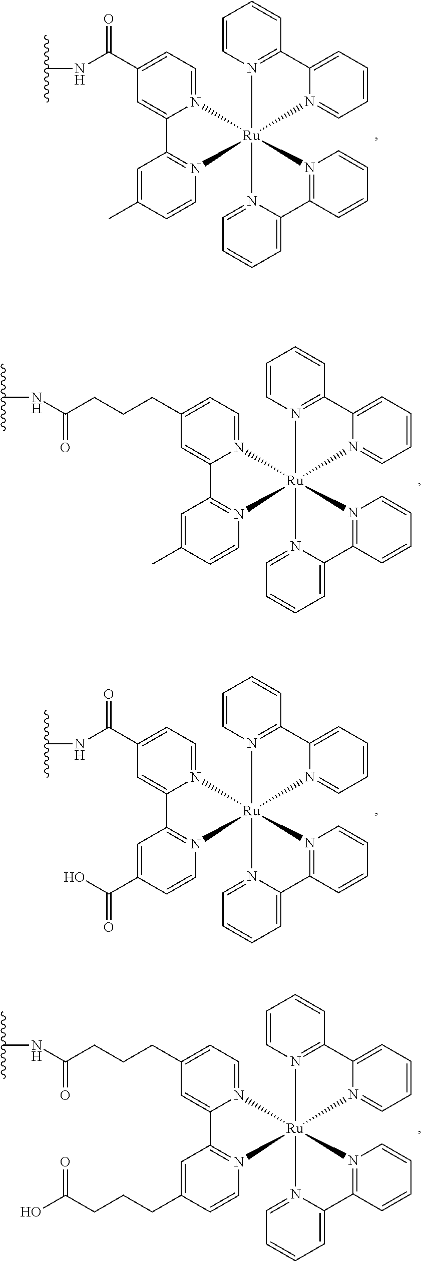 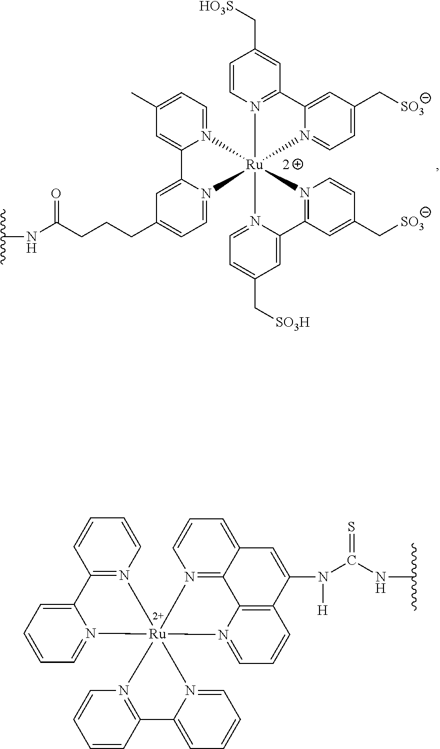
or a derivative thereof.

105. The photoreactive kit of claim 104, wherein the photoreactive moiety includes the moiety of

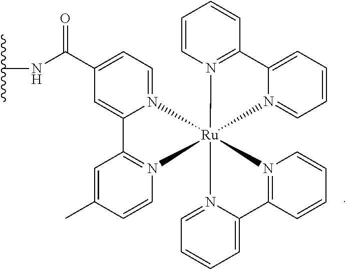

106. The photoreactive kit of claim 94, wherein the linker includes at least one of an amino acid, (PEG) n, an oligonucleotide, or a peptide, wherein n is an integer from 1 to 20.

107. The photoreactive kit of claim 94, wherein the photoreactive moiety is activatable at either a wavelength ranging from 700 nm to 1100 nm with a two-photon light source or a wavelength ranging from 200 nm to 1100 nm with a single light source so as to allow the primary subject probe to form the covalent bond with the nearby molecule in proximity to the target molecule in the sample.

108. The photoreactive kit of claim 94, wherein the primary subject probe is desthiobiotin-phenol or biotin-phenol.

109. The photoreactive kit of claim 94, further comprising:
a connector, wherein the connector is conjugatable with the detectable tag portion of the primary subject probe;
a tag-enzyme complex, wherein the tag of the tag-enzyme complex is conjugatable with the connector, and further wherein the enzyme of the tag-enzyme complex comprises peroxidase; and
an additional subject probe configured to form an additional subject probe covalent bond with the target molecule by catalytic activity of the peroxidase of the tag-enzyme complex.

110. The photoreactive kit of claim 94, wherein a concentration of the photoreactive probe ranges from 0.1 ug/mL to 100 ug/mL and a concentration of the primary subject probe ranges from 1 uM to 20 mM.

111. A method for photoreactive labeling, the method comprising:
delivering a plurality of photoreactive probes to a sample, wherein the photoreactive probes are represented by formula (I): 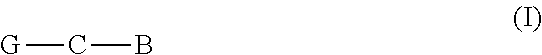
wherein
the C portion is a single chemical bond or a linker;
the B portion includes at least one photoreactive moiety bound to the C portion; and
the G portion comprises a bait molecule bound to the C portion;
non-covalently conjugating the bait molecules of a first portion of the photoreactive probe to a plurality of a target molecules in the sample;
delivering a plurality of primary subject probes to the sample, wherein the primary subject probes comprise a detectable tag portion bound to a photoexcitable subject moiety;
selectively illuminating a selected region of interest of the sample with optical radiation to thereby activate the photoreactive moieties of a plurality of photoreactive probes to form a plurality of photoactivated photoreactive probes in the selected region;
photoexciting, with the plurality of photoactivated photoreactive probes, the photoexcitable subject moieties of the primary subject probes to form a plurality of photoexcited primary subject probes with photoexcited subject moieties; and
forming covalent bonds between the plurality of photoexcited primary subject probes and a plurality of nearby molecules in proximity to the target molecules in the selected region of interest in the sample to thereby bind the plurality of primary subject probes to the plurality of nearby molecules in proximity to the target molecules.

112. An analytical method comprising:
delivering a plurality of photoreactive probes to the sample, wherein the plurality of photoreactive probes are represented by formula (I): 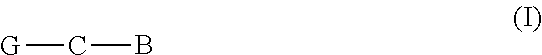
wherein
the C portion is a single chemical bond or a linker;
the B portion includes at least one photoreactive moiety bound to the C portion; and
the G portion comprises a bait molecule bound to the C portion;
non-covalently conjugating the bait molecules of a first portion of the photoreactive probe to a plurality of first molecules in the sample;
delivering a plurality of primary subject probes to the sample, wherein the primary subject probes comprise a detectable tag portion bound to a photoexcitable subject moiety; and
illuminating the sample from an imaging lighting source of an image-guided system;
imaging the illuminated sample with a camera;
acquiring with the camera at least one image of subcellular morphology of the sample in a first field of view;
processing the at least one image and determining a region of interest in the sample based on the processed image;
obtaining coordinate information of the region of interest;
and according to coordinate information, selectively illuminating the region of interest with optical radiation to activate the photoreactive moiety, wherein the activated photoreactive moiety allows the primary subject probe to form a covalent bond with the sample in the region of interest.
